SEQUENCE LISTING (94 sequences)

SEQ ID NO: 1 - DNA, 45 bp, Artificial sequence
  probe
gacgaggtgggggtgcccgaagccatagccgacaacctgactgtg

SEQ ID NO: 2 - DNA, 45 bp, Artificial sequence
  probe
ggagatgccagcggttttcgacaatgtaaagatcctgggagactt

SEQ ID NO: 3 - DNA, 45 bp, Artificial sequence
  probe
gcactccttgagcctctcccccttgaccctcatcttcttgacaag

SEQ ID NO: 4 - DNA, 45 bp, Artificial sequence
  probe
atttgcttgccgtagcccgatgaagcccactcgcagtttgaggac

SEQ ID NO: 5 - DNA, 45 bp, Artificial sequence
  probe
gcactctgcttcggtctacgccacgcaaaagatggactttgagag

SEQ ID NO: 6 - DNA, 45 bp, Artificial sequence
  probe
agatctctccgggtggctcctgttgaccggggtggccgtccagtt

SEQ ID NO: 7 - DNA, 60 bp, Artificial sequence
  probe
gccttttctctgcctcattcttgtattgtttgttgtattgtgaaataaattgctcgaaat

SEQ ID NO: 8 - DNA, 45 bp, Artificial sequence
  probe
gttgatgtcggagtagtcgaacgcgaccgggttgtcctccaccat

SEQ ID NO: 9 - DNA, 60 bp, Artificial sequence
  probe
cgacacggacacggattaattaataagcattaaaatgtttcacttgaacgaggcctttat

SEQ ID NO: 10 - DNA, 45 bp, Artificial sequence
  probe
attgttgagagagtcaaagtaggcggtgagaaaggagagggacag

SEQ ID NO: 11 - DNA, 46 bp, Artificial sequence
  probe
agcacgacaggaacgactccaacgacgcagagaaacacaagtataa

SEQ ID NO: 12 - DNA, 49 bp, Artificial sequence
  probe
gatacaaaaccgaggattggacactgcaagacacatgcgaggaactatg

SEQ ID NO: 13 - DNA, 50 bp, Artificial sequence
  probe
aaataaggtatcagaggaaggggactggaagcccatagtcaggtttctaa

SEQ ID NO: 14 - DNA, 45 bp, Artificial sequence
  probe
tagacctaaacagtccagaggagcaggacgacaatggaaacactg

SEQ ID NO: 15 - DNA, 45 bp, Artificial sequence
  probe
ctatagcacggaggcatggaccctacgagacacgtgtctggagat

SEQ ID NO: 16 - DNA, 45 bp, Artificial sequence
  probe
ctctacgcgaacggtcggagagggggaagtggagtgttacaacaa

SEQ ID NO: 17 - DNA, 47 bp, Artificial sequence
  probe
acacatctgcttgtgctactgctcttcctgtggctctctcaactaac

SEQ ID NO: 18 - DNA, 50 bp, Artificial sequence
  probe
aaataagtttggcctgcccgatgctgatttgtatgatccagatacccaga

SEQ ID NO: 19 - DNA, 59 bp, Artificial sequence
  probe
gtccaagaaatagaagcgctactaaaccatgtaaatcagcaccgtataaagttcctcac

SEQ ID NO: 20 - DNA, 45 bp, Artificial sequence
  probe
aatcaagcagaggggggtaacgacacacatctcaagtggggctac

SEQ ID NO: 21 - DNA, 50 bp, Artificial sequence
  probe
gtttgtgtttccgtctctctatccatttcccccaccaatacctcaactat

SEQ ID NO: 22 - DNA, 45 bp, Artificial sequence
  probe
tatagcagcgcccgatgccaacgcgacagggtgagtcacatagat

SEQ ID NO: 23 - DNA, 57 bp, Artificial sequence
  probe
gtgtaacttttatggtgtctgtatgcagttttgtagagttttttgttacggggggag

SEQ ID NO: 24 - DNA, 60 bp, Artificial sequence
  probe
caatacagaggttgtgtttggcaaaatttaagcagcaattgttgtatctaaatattaagg

SEQ ID NO: 25 - DNA, 51 bp, Artificial sequence
  probe
ttagcaatttttaaggttctaacccgcttgggtaggaaagaccctaaccac

SEQ ID NO: 26 - DNA, 48 bp, Artificial sequence
  probe
atttgcatgccgatcacgtggtctctcccctaacacataatagccttt

SEQ ID NO: 27 - DNA, 48 bp, Artificial sequence
  probe
aaaggctattatgtgttaggggagagaccacgtgatcggcatgcaaat

SEQ ID NO: 28 - DNA, 45 bp, Artificial sequence
  probe
atcttggtggtgttctgcgccaggaagcccaggatggtgttgaag

SEQ ID NO: 29 - DNA, 45 bp, Artificial sequence
  probe
cgcggatcatcagggactccatttcgaaatcgatcgactcgttcg

SEQ ID NO: 30 - DNA, 45 bp, Artificial sequence
  probe
aaacccctcgagccgatcctcgtccgtgtcgctgttccagaacca

SEQ ID NO: 31 - DNA, 45 bp, Artificial sequence
  probe
ccgacgcggtgcaggctctcgacctggacgatgaggcggtggaag

SEQ ID NO: 32 - DNA, 45 bp, Artificial sequence
  probe
cgactgacagtctagccgcaggtcgtggttgttcatcagccactg

SEQ ID NO: 33 - DNA, 45 bp, Artificial sequence
  probe
gcgaggagcagcaggatcaggtcggcgtgtccccacgcgtccgcg

SEQ ID NO: 34 - DNA, 45 bp, Artificial sequence
  probe
ctgcgtcacctgccggcgcgcgcgggcgtggcgggccgttaaaag

SEQ ID NO: 35 - DNA, 45 bp, Artificial sequence
  probe
gaggaagtgtgcgcggaagacccaccacgtggacgccgaccgcgc

SEQ ID NO: 36 - DNA, 45 bp, Artificial sequence
  probe
ctacgcctcgaccgcgcaccaccggggcgtcgtgttctacctcac

SEQ ID NO: 37 - DNA, 45 bp, Artificial sequence
  probe
cacggcgtgcccctcaacaccgactactacttctcgcacctcatc

SEQ ID NO: 38 - DNA, 45 bp, Artificial sequence
  probe
aagacgatctgcggcacgaacctagaccgggcgttctccacagcc

SEQ ID NO: 39 - DNA, 45 bp, Artificial sequence
  probe
aaagaggggggcggtggcgcgacagaagcgctcctggcgctcgac

SEQ ID NO: 40 - DNA, 45 bp, Artificial sequence
  probe
cgacgattcccggaaggacgatgtgctgcgctctcgcctttatac

SEQ ID NO: 41 - DNA, 45 bp, Artificial sequence
  probe
gcgcggctgcgtgagcgtgaagttcacgcccccgcccgtctcgtg

SEQ ID NO: 42 - DNA, 45 bp, Artificial sequence
  probe
acttaatagccgacgccggggcgcgggggaccttatttcgaattc

SEQ ID NO: 43 - DNA, 45 bp, Artificial sequence
  probe
gaagcgcccgaagagccggaagccctcgccctgggcgtcgctgag

SEQ ID NO: 44 - DNA, 45 bp, Artificial sequence
  probe
catcggctgcgggaggaggttctggtagatccggggggtggggat

SEQ ID NO: 45 - DNA, 45 bp, Artificial sequence
  probe
gaggacgcgggcatcgacatcgtcgtccacaagcgcgtggagatc

SEQ ID NO: 46 - DNA, 45 bp, Artificial sequence
  probe
gacctgccgtctctgcggcgcagcatcgacgccacgtttcgcctc

SEQ ID NO: 47 - DNA, 52 bp, Artificial sequence
  probe
ctgctcttctgccttctaccaacaggtaaactacactactgcactgcaaaaa

SEQ ID NO: 48 - DNA, 54 bp, Artificial sequence
  probe
agagaaacacgtcacaggcaaagtcatgtaaaacccgggttaaaaatagcctaa

SEQ ID NO: 49 - DNA, 45 bp, Artificial sequence
  probe
acgaaacagccgaacgtttcctaatcacgcccatggaaccatagc

SEQ ID NO: 50 - DNA, 45 bp, Artificial sequence
  probe
caaggtgactgaaaaggtcatagagaggacaccggtcccatttcc

SEQ ID NO: 51 - DNA, 47 bp, Artificial sequence
  probe
ttttacctgtatatgcgtatgaagcaatcggaccccagtggtttcgc

SEQ ID NO: 52 - DNA, 45 bp, Artificial sequence
  probe
cagcatgggggcgtttggatccggtagggcaccgggctgaaattt

SEQ ID NO: 53 - DNA, 45 bp, Artificial sequence
  probe
gtccaaggcaccctgggtcctcttacgaatgtctgactacttcag

SEQ ID NO: 54 - DNA, 48 bp, Artificial sequence
  probe
gttgatggcattgagaagcaggtggtctaatgtcatgcgcatagtctg

SEQ ID NO: 55 - DNA, 45 bp, Artificial sequence
  probe
gaggcctgcaaacgcttggggctttcgtggaggaacaaatgacgt

SEQ ID NO: 56 - DNA, 45 bp, Artificial sequence
  probe
actgcctggagaattttccaccccctctaccatttcgcgccttta

SEQ ID NO: 57 - DNA, 45 bp, Artificial sequence
  probe
gtaagagggagacccaaaggcggcggcactaaagattgttctggt

SEQ ID NO: 58 - DNA, 48 bp, Artificial sequence
  probe
gacttaataaggctcaaaaaacacagtgacccaagcatggcgtcgaac

SEQ ID NO: 59 - DNA, 45 bp, Artificial sequence
  probe
cagcggatgtgttttgcccaaacacgagggttgcaggaaacaggt

SEQ ID NO: 60 - DNA, 45 bp, Artificial sequence
  probe
gtgaaggaatgggagccaccggtaaagtaggactagacacaaatg

SEQ ID NO: 61 - DNA, 45 bp, Artificial sequence
  probe
cccacggggacatggacaggggtgttaggtctggtggaacatgct

SEQ ID NO: 62 - DNA, 45 bp, Artificial sequence
  probe
cgcccccggggacaccctctgtagtcagacttgggttagggcttt

SEQ ID NO: 63 - DNA, 49 bp, Artificial sequence
  probe
gaaaaacttgaacagcgaaaccctctcttccgcgacttcttcgattttg

SEQ ID NO: 64 - DNA, 45 bp, Artificial sequence
  probe
acattcccgcacctttgcctgtagccaggcaaccttccgtttgat

SEQ ID NO: 65 - DNA, 52 bp, Artificial sequence
  probe
cttctaaactgtcgtttgatgcactagacgcacccccgactcaaattataga

SEQ ID NO: 66 - DNA, 50 bp, Artificial sequence
  probe
gaagttgttaaagtttcccccgtcccactggctatattagttaccgcaac

SEQ ID NO: 67 - DNA, 47 bp, Artificial sequence
  probe
attgggcagacatgtgagcaataaggaacgtgggctgctagatacaa

SEQ ID NO: 68 - DNA, 58 bp, Artificial sequence
  probe
atagagaggtggaaacacacaaacaggcagactctaggaagtgtggtttagtatatag

SEQ ID NO: 69 - DNA, 45 bp, Artificial sequence
  probe
gaacagaccgactccgggcgcgaggaggacgcacaggagagcgag

SEQ ID NO: 70 - DNA, 45 bp, Artificial sequence
  probe
cgtccaccgtccctctcacccccactcgaatcgcgcaggcgcgtc

SEQ ID NO: 71 - DNA, 45 bp, Artificial sequence
  probe
atcacgaagtgtcaactcccgcatctccgtcgcgcaacgtgtcct

SEQ ID NO: 72 - DNA, 49 bp, Artificial sequence
  probe
cattttaacacctcggaaacggtcgaatgatcaaggtcaatcctcaacg

SEQ ID NO: 73 - DNA, 50 bp, Artificial sequence
  probe
gtttctgttcatccgcccgtgactgaacagtatcaaatgctaccgtattc

SEQ ID NO: 74 - DNA, 60 bp, Artificial sequence
  probe
ccccgagaatgtttcattttaggatcattagtgtcttttgggtcatgatgttttttgtaa

SEQ ID NO: 75 - DNA, 60 bp, Artificial sequence
  probe
tgcaaaggcagcaatgttagcaaaatttaaagagttatacggggtgagtttttcagaatt

SEQ ID NO: 76 - DNA, 49 bp, Artificial sequence
  probe
caacgaagtatcctctcctgaaattattaggcagcacttggccaaccac

SEQ ID NO: 77 - DNA, 45 bp, Artificial sequence
  probe
ccttgggcaccgaagaaacacagacgactatccagcgaccaagat

SEQ ID NO: 78 - DNA, 45 bp, Artificial sequence
  probe
ggaattttggtctacaacctcccccaggaggcacactagaagata

SEQ ID NO: 79 - DNA, 45 bp, Artificial sequence
  probe
aaaagggactgcccacaactcaaaccccctcaggaggaaggggaa

SEQ ID NO: 80 - DNA, 46 bp, Artificial sequence
  probe
caacacccagttcaaactcctccaaacccccctacacatattcttg

SEQ ID NO: 81 - DNA, 46 bp, Artificial sequence
  probe
ccttccaatttcccaggaaaaaacacaacaaaccccaggccaaata

SEQ ID NO: 82 - DNA, 45 bp, Artificial sequence
  probe
cttctcccccattggtccatgactccgaccttgaacatgtcctaa

SEQ ID NO: 83 - DNA, 49 bp, Artificial sequence
  probe
cagacaggcataaccattctcgctctactcctcctcgtcatattgtttg

SEQ ID NO: 84 - DNA, 45 bp, Artificial sequence
  probe
ctttctgcgccgctcccgctcctcacgctcctgcagaagttttaa

SEQ ID NO: 85 - DNA, 45 bp, Artificial sequence
  probe
ctgcagaagttttaagatctcccgctgctcctccgccaacagtct

SEQ ID NO: 86 - DNA, 45 bp, Artificial sequence
  probe
cactcctgtctcccccaaggttccacctgccttctttcaatcaat

SEQ ID NO: 87 - DNA, 53 bp, Artificial sequence
  probe
tataacccttactgtattgacatacagcaatggggctacacgggatctttgag

SEQ ID NO: 88 - DNA, 45 bp, Artificial sequence
  probe
gcgaaggaaaaggcggaatcgtctgccgccgccgtcgatcccttt

SEQ ID NO: 89 - DNA, 45 bp, Artificial sequence
  probe
gtatgtccgcagatgttgaaaaaggtcaggaacaccagcaggacg

SEQ ID NO: 90 - DNA, 45 bp, Artificial sequence
  probe
atcatggtggacgtgggaggcacgggtggtttaataatgatcttg

SEQ ID NO: 91 - DNA, 45 bp, Artificial sequence
  probe
atcgtgagggacgacgatagacgaaaactcacctcggtctgttaa

SEQ ID NO: 92 - DNA, 45 bp, Artificial sequence
  probe
cttgcgggaaatcacagcgtaggtgaggctcccaactggcatcgg

SEQ ID NO: 93 - DNA, 47 bp, Artificial sequence
  probe
gttgacgaccccgctgctctcaaaatcccgtagatgtgtatcttttt

SEQ ID NO: 94 - DNA, 60 bp, Artificial sequence
  probe
aggaagcggaacagtaaataaagttcttagaaaagttttttactctgttttagggggtgg

(19) United States
(12) Patent Application Publication
(10) Pub. No.: US 2018/0291463 A1
(43) Pub. Date: Oct. 11, 2018

(54) Compositions and Methods for Detecting the Ovarian Cancer Oncobiome

(71) Applicant: THE TRUSTEES OF THE UNIVERSITY OF PENNSYLVANIA, Philadelphia, PA (US)

(72) Inventors: Erle S. Robertson, Wynnewood, PA (US); James C. Alwine, Tucson, AZ (US)

(21) Appl. No.: 15/941,723

(22) Filed: Mar. 30, 2018

Related U.S. Application Data
(60) Provisional application No. 62/601,816, filed on Mar. 31, 2017.

(51) Int. Cl. C12Q 1/6886; C12Q 1/6874

(52) U.S. Cl. CPC C12Q 1/6886; C12Q 1/6874

(57) ABSTRACT

The present invention includes compositions and methods for the detection of ovarian cancer. Compositions and methods are provided for detecting a metagenomic signature in a tissue sample from a subject that indicates the subject has ovarian cancer.

### CROSS-REFERENCE TO RELATED APPLICATION

The present application is entitled to priority under 35 U.S.C. § 119(e) to U.S. Provisional Patent Application No. 62/601,816, filed Mar. 31, 2017, which is hereby incorporated by reference in its entirety herein.

### BACKGROUND OF THE INVENTION

In the US, ovarian cancer is the second most common and deadliest among gynecologic cancers (affecting about 1/70), with a mortality rate of 1% of all women. It is the 5th leading cause of cancer-related deaths in women, causing an estimated 22,280 new cases (1.3% of all new cancer cases) and 14,240 deaths (2.4% of all cancer deaths) by 2016. Importantly, the incidence is even higher in developed countries. Due to the asymptomatic nature of the early stage of the disease most patients are diagnosed at an advanced stage. Thus finding specific biomarkers for early diagnosis of the disease is of utmost importance. Many studies have found that DNA of the Human Papillomavirus (HPV)-16 and HPV-18 is associated with ovarian carcinomas. However, recent studies have found that the tumor microbiome may be far more complex. Unique microbial signatures associated with triple negative breast cancer and head and neck cancer have been defined. These signatures potentially provide insight into predisposition, presence or prognosis of the cancer. Such diagnostic data may increase the therapeutic potential for early detection and treatment.

PathoChip is a microarray-based approach which comprises probes for detection of all known viruses and other human pathogenic microorganisms (Banerjee et al. Sci Rep. 2015; 5:15162; Baldwin et al. MBio. 2014; 5: e01714-14). The current version of the PathoChip contains 60,000 probes representing all known viruses, 250 helminths, 130 protozoa, 360 fungi and 320 bacteria. In addition to probes specific for the viruses and micro-organisms, PathoChip also contains family-specific conserved probes which provide a means for detecting previously uncharacterized members of a family.

A need exists for compositions and methods for detection and treatment of ovarian cancer. The present invention satisfies this need.

### SUMMARY OF THE INVENTION

As described herein, the present invention relates to compositions and methods for detecting ovarian cancer.

In one aspect, the invention includes a method of detecting ovarian cancer in a tumor tissue sample from a subject. In certain embodiments, the method comprises hybridizing a detectably-labeled nucleic acid from the tumor tissue sample to a PathoChip array to generate a first hybridization pattern and hybridizing a detectably-labeled nucleic acid from a reference sample to a PathoChip array to generate a second hybridization pattern. In certain embodiments, the reference sample is from an otherwise identical non-tumor tissue from a subject. In some embodiments the first and second hybridization patterns are compared and when the first hybridization pattern is substantially a microbial hybridization signature and the second hybridization pattern is substantially not a microbial hybridization signature, ovarian cancer is detected in the tumor tissue sample.

In another aspect, the invention includes a method of detecting ovarian cancer in a tumor tissue sample from a subject. In certain embodiments, the method comprises hybridizing a detectably-labeled nucleic acid from the tumor tissue sample to a first microarray comprising at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria to generate a first hybridization pattern. In certain embodiments, a detectably-labeled nucleic acid from a reference sample is hybridized to a second microarray comprising at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria to generate a second hybridization pattern. In certain embodiments, the reference sample is from an otherwise identical non-tumor tissue from a subject. In certain embodiments, the first and second hybridization patterns are compared and when the first hybridization pattern is substantially a microbial hybridization signature and the second hybridization pattern is substantially not a microbial hybridization signature, ovarian cancer is detected in the tumor tissue sample.

Another aspect of the invention includes a composition comprising at least three nucleic acid probes selected from the group consisting of SEQ ID NOS: 1-94.

Yet another aspect of the invention includes a microarray comprising at least three nucleic acid probes selected from the group consisting of SEQ ID NOS: 1-94.

Still another aspect of the invention includes a microarray comprising at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

One aspect of the invention includes a kit comprising at least three nucleic acid probes selected from the group consisting of SEQ ID NOS: 1-94, and instructional material for use thereof.

Another aspect of the invention includes a kit comprising a microarray comprising at least three nucleic acid probes selected from the group consisting of SEQ ID NOS: 1-94, and instructional material for use thereof.

Yet another aspect of the invention includes a kit comprising a microarray comprising at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptomphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

In various embodiments of the above aspects or any other aspect of the invention delineated herein, the microbial hybridization signature is generated by hybridization of the detectably-labeled nucleic acid from the tumor tissue sample to at least three nucleic acid probes on the PathoChip. In certain embodiments, the probes are from microbes selected from the group consisting of: Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptomphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

In certain embodiments, the nucleic acid probes are selected from the group consisting of SEQ ID NOs: 1-94.

In other embodiments, the tumor tissue sample is selected from the group consisting of a biopsy, formalin-fixed, paraffin-embedded (FFPE) sample, or non-solid tumor.

In certain embodiments, the subject is human. In other embodiments, the method further comprises wherein when oral ovarian cancer is detected in the tumor tissue sample from a subject, the subject is provided with a treatment for ovarian cancer. In yet another embodiment, the treatment comprises surgery, chemotherapy, or radiotherapy.

In certain embodiments, the detectably-labeled nucleic acid is labeled with a fluorophore, radioactive phosphate, biotin, or enzyme. In another embodiment, the fluorophore is Cy3 or Cy5.

In certain embodiments, the nucleic acid probes are selected from about 10 to about 30 microbes and comprise about 3 to about 5 probes per microbe.

In yet other embodiments, the microarray is a biochip, glass slide, bead, or paper.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1G are a series of plots illustrating viral signatures detected in ovarian, matched and non-matched controls. FIG. 1A displays molecular signatures of viral groups detected in ovarian cancer, with the total hybridization signal for each viral groups represented according to descending order as a bar graph and prevalence of the same as dots. FIG. 1B is a pie chart displaying tumorigenic viral signatures detected in the ovarian cancers. FIG. 1C is a bar graph showing the average hybridization signal of the tumorigenic viral signatures detected in the ovarian cancers represented in decreasing order, whereas their respective prevalence are represented as dots. FIGS. 1D-1E show the signatures of viral families detected in matched (FIG. 1D) and non-matched (FIG. 1E) controls; represented according to decreasing average hybridization signals as bar graphs, and their respective prevalence as dots. FIG. 1F is a heat map of average hybridization signals for probes of Poxviruses, Retroviruses, Herpesviruses, Polyomaviruses and Papillomaviruses detected in ovarian cancers (OC), matched (MC) and non-matched (NC) controls. Heat map of average hybridization signal of both conserved and specific probes of Poxviridae are shown. Among the conserved poxviridae probes mentioned, (a) comprises the conserved probes detected significantly in the ovarian cancer versus the controls, and (b) comprises the conserved probes detected significantly in the controls versus the ovarian cancers screened. In the heat map with Herpesviridae probes, those mentioned (c) are conserved probes. All other probes in these heat maps are specific probes. FIG. 1G is a Venn diagram showing the number of viral families common or unique to the ovarian cancer and control samples.

FIGS. 2A-2C are a series of charts illustrating bacterial signatures detected in ovarian, matched and non-matched controls. FIG. 2A shows bacterial signatures detected in ovarian cancers, matched and non-matched controls. The prevalence of those signatures are represented in the decreasing order as dots, and their average hybridization signal being represented as a bar graph. FIG. 2B shows the distribution of bacterial phyla detected in ovarian cancer, matched and non-matched controls. FIG. 2C is a Venn diagram showing the number of bacteria common or unique to the ovarian cancer and control samples.

FIGS. 3A-3B are a series of graphs illustrating fungal signatures detected in ovarian, matched and non-matched controls. FIG. 3A depicts fungal signatures detected in ovarian cancer, matched and non-matched controls. The prevalence of those signatures are represented in the decreasing order as dots, and their average hybridization signal being represented as a bar graph. FIG. 3B is a Venn diagram showing the number of fungi common or unique to the ovarian cancer and control samples.

FIGS. 4A-4B are a series of graphs illustrating parasitic signatures detected in ovarian, matched and non-matched controls. FIG. 4A depicts parasitic signatures detected in ovarian cancer, matched and non-matched controls. The prevalence of those signatures are represented in the decreasing order as dots, and their average hybridization signal being represented as a bar graph. FIG. 4B is a Venn diagram showing the number of parasites common or unique to the ovarian cancer and control samples.

FIGS. 5A-5C are a series of plots and images illustrating hierarchical clustering of ovarian cancer samples screened. Hierarchical clustering of 100 oral cancer samples. FIG. 5A shows hierarchical clustering by R program using Euclidean distance, complete linkage and non-adjusted values. Samples marked (′) were the samples that were screened in pools, the rest were screened individually. FIG. 5B shows clustering of the OSCC samples using NBCIust software [CH (Calinski and Harabasz) index, Euclidean distance, complete linkage]. FIG. 5C shows topological analysis using Ayasdi software, using Euclidean (L2) metric and L-infinity centrality lenses. The cancer samples that had similar detection for viral and microbial signatures formed the nodes, and those nodes are connected by an edge if the corresponding node have detection pattern in common to the first node. Each nodes are colored according to the number of samples clustered in each node.

FIGS. 6A-6B are a series of images illustrating targeted MiSeq reads align to capture probe locations. Probe capture sequencing alignment is shown for individual capture pools (Capture 1-6 or, C1-6). The whole genome amplified DNA plus cDNA of the ovarian cancer samples were hybridized to a set of biotinylated probes, then captured by streptavidin beads, and used for tagmentation, library preparation and deep sequencing with paired-end 250-nt reads. The total number of MiSeq reads per capture pool for HPV18 (FIG. 6A) and Yaba Monkey Tumor Virus (FIG. 6B) are mentioned at the right end of the read coverage track. For example 302 reads were obtained for C2 capture. The Miseq reads from individual capture when aligned with the metagenome of PathoChip (Chip probes) was found to cluster mostly at the capture probe regions. The genomic locations are mentioned in the figure for each organism. FIG. 6A shows the MiSeq read alignment to the HPV18 probes on the PathoChip. The probes corresponding to the HPV18 genes are mentioned. It also shows the heat map of hybridization signals of all the HPV18 probes in the PathoChip with the ovarian samples. The HPV18 probes marked (*) are the probes that were biotinylated and used for capture of the HPV18 sequences from the whole genome amplified DNA plus cDNA of the ovarian cancer samples. FIG. 6B shows the MiSeq read alignment to the PathoChip probes for Yaba Monkey Tumor Virus. MiSeq reads aligned to the 1 capture probe used which corresponded to g52R gene of the virus.

FIGS. 7A-7E are a series of plots and images illustrating viral genomic integrations in the host chromosome. FIG. 7A depicts alignment of the MiSeq reads to the reference of HHV6A, showing soft-clipped regions that do not align to the corresponding viral reference sequences. These soft-clipped reads shown were then extracted from the alignment and mapped (containing sequences of potential pathogen-integrated human loci) to the human genome, which reveals the exact human and pathogen integration breakpoints. FIG. 7B is a karyogram plot of virus insertion sites in human chromosomes. All the insertion sites were included. The number of insertion sites in each chromosome is mentioned in the figure before each chromosome number. G-banding annotation for each chromosome is shown; gneg—Giemsa negative bands; The Giemsa positive bands have further been subdivided into gpos25, gpos50, gpos75, and gpos100 with the higher number indicating a darker stain; acen—centromeric regions; gvar—variable length heterochromatic regions; stalk—tightly constricted regions on the short arms of the acrocentric chromosomes. FIG. 7C is a Circos plot highlighting fusion events for the viral insertions into individual human chromosomes. All the reads were taken into account and chromosome numbers are mentioned. Viral insertions for individual families are represented in the inner concentric circular tracks. The outermost track shows all the insertions taken together highlighting the karyotype of each chromosome. FIG. 7D shows the number of individual viral genomic insertions in human somatic chromosomes detected in the study. FIG. 7E depicts the association of host genes affected by viral genomic integrations to malignant tumor formation, analyzed by Ingenuity Pathway Analysis (IPA) program that showed highly significant p-value for such association.

FIGS. 8A-8B are a table displaying the microbial signatures detected in OSCC and control samples.

FIG. 9 is a set of bar graphs illustrating molecular signatures of viral families detected in ovarian cancer represented according to decreasing average hybridization signal and prevalence.

FIGS. 10A-10D are a series of images illustrating probe capture sequencing alignments post MiSeq. The MiSeq reads from individual capture (C1-6) when aligned with the metagenome of PathoChip (Chip probes) was found to cluster mostly at the capture probe regions. The genomic location along with the number of MiSeq reads are noted.

FIGS. 11A-11C show the available clinical details of the 99 ovarian cancer samples screened.

FIGS. 12A-12C show the statistical significance between ovarian cancer samples of Clusters 1, 2 and 3 obtained by NBClust software.

FIGS. 13A-13B show the statistical significance between ovarian cancer samples of Groups A, B, C and singletons that are obtained by topological-based data analyses using Ayasdi software.

FIGS. 14A-14F are a list of capture probe sequences used for capture sequencing.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, exemplary materials and methods are described herein. In describing and claiming the present invention, the following terminology will be used.

It is also to be understood that the terminology used herein is for the purpose of describing particular embodiments only, and is not intended to be limiting.

The articles “a”, “an”, and “the” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “biomarker” or “marker” as used herein generally refers to a nucleic acid molecule, clinical indicator, protein, or other analyte that is associated with a disease. In certain embodiments, a nucleic acid biomarker is indicative of the presence in a sample of a pathogenic organism, including but not limited to, viruses, viroids, bacteria, fungi, helminths, and protozoa. In various embodiments, a marker is differentially present in a biological sample obtained from a subject having or at risk of developing a disease (e.g., an infectious disease) relative to a reference. A marker is differentially present if the mean or median level of the biomarker present in the sample is statistically different from the level present in a reference. A reference level may be, for example, the level present in an environmental sample obtained from a clean or uncontaminated source. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing a disease (e.g., an infectious disease), for determining the prognosis of the subject, for evaluating therapeutic efficacy, or for selecting a treatment regimen.

By “agent” is meant any nucleic acid molecule, small molecule chemical compound, antibody, or polypeptide, or fragments thereof.

By “alteration” or “change” is meant an increase or decrease. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

By “capture reagent” is meant a reagent that specifically binds a nucleic acid molecule or polypeptide to select or isolate the nucleic acid molecule or polypeptide.

As used herein, the terms “determining”, “assessing”, “assaying”, “measuring” and “detecting” refer to both quantitative and qualitative determinations, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable moiety” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

By “fragment” is meant a portion of a nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 10, 15, 20, 30, 40, 50, 60, 70, 80, 90, or 100 nucleotides.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Hybridization” means hydrogen bonding, which may be Watson-Crick, Hoogsteen or reversed Hoogsteen hydrogen bonding, between complementary nucleobases. For example, adenine and thymine are complementary nucleotides that pair through the formation of hydrogen bonds.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “marker profile” is meant a characterization of the signal, level, expression or expression level of two or more markers (e.g., polynucleotides).

By the term “microbe” is meant any and all organisms classed within the commonly used term “microbiology,” including but not limited to, bacteria, viruses, fungi and parasites.

By the term “microarray” is meant a collection of nucleic acid probes immobilized on a substrate. As used herein, the term “nucleic acid” refers to deoxyribonucleotides, ribonucleotides, or modified nucleotides, and polymers thereof in single- or double-stranded form. The term encompasses nucleic acids containing known nucleotide analogs or modified backbone residues or linkages, which are synthetic, naturally occurring, and non-naturally occurring. Nucleic acid molecules useful in the methods of the invention include any nucleic acid molecule that specifically binds a target nucleic acid (e.g., a nucleic acid biomarker). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

“Parenteral” administration of an immunogenic composition includes, e.g., subcutaneous (s.c.), intravenous (i.v.), intramuscular (i.m.), or intrasternal injection, or infusion techniques.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By “reference” is meant a standard of comparison. As is apparent to one skilled in the art, an appropriate reference is where an element is changed in order to determine the effect of the element. In one embodiment, the level of a target nucleic acid molecule present in a sample may be compared to the level of the target nucleic acid molecule present in a clean or uncontaminated sample. For example, the level of a target nucleic acid molecule present in a sample may be compared to the level of the target nucleic acid molecule present in a corresponding healthy cell or tissue or in a diseased cell or tissue (e.g., a cell or tissue derived from a subject having a disease, disorder, or condition).

As used herein, the term “sample” includes a biologic sample such as any tissue, cell, fluid, or other material derived from an organism.

By “specifically binds” is meant a compound (e.g., nucleic acid probe or primer) that recognizes and binds a molecule (e.g., a nucleic acid biomarker), but which does not substantially recognize and bind other molecules in a sample, for example, a biological sample.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95%, 96%, 97%, 98%, or even 99% or more identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By the term “substantially microbial hybridization signature” is a relative term and means a hybridization signature that indicates the presence of more microbes in a tumor sample than in a reference sample. By the term “substantially not a microbial hybridization signature” is a relative term and means a hybridization signature that indicates the presence of less microbes in a reference sample than in a tumor sample.

By “subject” is meant a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

By “target nucleic acid molecule” is meant a polynucleotide to be analyzed. Such polynucleotide may be a sense or antisense strand of the target sequence. The term “target nucleic acid molecule” also refers to amplicons of the original target sequence. In various embodiments, the target nucleic acid molecule is one or more nucleic acid biomarkers.

A “target site” or “target sequence” refers to a genomic nucleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By the term “tumor tissue sample” is meant any sample from a tumor in a subject including any solid and non-solid tumor in the subject.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention features compositions and methods for the detection or diagnosis of ovarian cancer in a subject. Metagenomic signatures comprising detecting genetic material from a number of viral, bacterial, fungal, and parasitic microbes were identified that indicate that a subject has ovarian cancer.

As described herein, the ovarian cancer microbial signature was defined using 100 ovarian cancer samples and 20 matched and 20 unmatched control samples. This microbial signature pattern was significantly associated with the cancer samples and was distinct from the signature pattern detected in the controls. To corroborate these results microbial probes were selected across the different organisms positive in the PathoChip screen and used for hybrid-capture selection from the ovarian cancer samples. This enrichment and amplification allowed targeted next generation sequencing that validated the PathoChip screen results. The sequencing also allowed identification of microbial genomic insertions in the host chromosomes of the ovarian cancer tissues. The data generated in this study elucidate a robust and specific microbiome associated with ovarian cancer.

### Methods

The present invention includes methods of detecting ovarian cancer in a tumor tissue sample from a subject. In one aspect, the method comprises hybridizing a detectably-labeled nucleic acid from the tumor tissue sample to a PathoChip array to generate a first hybridization pattern, then hybridizing a detectably-labeled nucleic acid from a reference sample to a PathoChip array to generate a second hybridization pattern. The reference sample is from an otherwise identical non-tumor tissue from a subject. The first and second hybridization patterns are compared. When the first hybridization pattern is substantially a microbial hybridization signature and the second hybridization pattern is substantially not a microbial hybridization signature, ovarian cancer is detected in the tumor tissue sample.

In another aspect of the invention the method comprises wherein the microbial hybridization signature is generated by hybridization of the detectably-labeled nucleic acid from the tumor tissue sample to at least three nucleic acid probes on the PathoChip, wherein the probes are from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

Another aspect of the invention includes a method of detecting ovarian cancer in a tumor tissue sample from a subject comprising hybridizing a detectably-labeled nucleic acid from the tumor tissue sample to a first microarray. The first microarray comprises at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria. A first hybridization pattern is generated. Then, hybridizing a detectably-labeled nucleic acid from a reference sample to a second microarray. The second microarray comprises at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptomphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria. A second hybridization pattern is generated. The reference sample is from an otherwise identical non-tumor tissue from a subject. The first and second hybridization patterns are compared. When the first hybridization pattern is substantially a microbial hybridization signature and the second hybridization pattern is substantially not a microbial hybridization signature, ovarian cancer is detected in the tumor tissue sample.

In certain embodiments of the invention, the probes are selected from the group consisting of SEQ ID NOS: 1-94.

In the methods disclosed herein, the tumor tissue sample can be a biopsy, formalin-fixed, paraffin-embedded (FFPE) sample, or non-solid tumor. The detectably-labeled nucleic acid can be labeled with a fluorophore, radioactive phosphate, biotin, or enzyme and the fluorophore can be Cy3 or Cy5.

The methods can also include providing the subject with a treatment for ovarian cancer when ovarian is detected in the tumor tissue sample from the subject. Examples of treatments include, but are not limited to, surgery, chemotherapy, or radiotherapy. The subject can be any human or non-human mammal, such as a bovine, equine, canine, ovine, feline, mouse, or monkey. In one embodiment, the subject is a human.

### Target Nucleic Acid Molecules

Methods and compositions of the invention are useful for the identification of a target nucleic acid molecule in a biological sample to be analyzed. Target sequences are amplified from any biological sample that comprises a target nucleic acid molecule. Such samples may comprise fungi, spores, viruses, or cells (e.g., prokaryotes, eukaryotes, including human). Such samples may comprise viral, bacterial, fungal, and parasitic nucleic acid molecules. In specific embodiments, compositions and methods of the invention detect one or more nucleic acid sequences from one or more pathogenic organisms, including viruses, viroids, bacteria, fungi, helminths, and/or protozoa.

In one embodiment, a sample is a biological sample, such as a tissue or tumor sample. The level of one or more polynucleotide biomarkers (e.g., to detect or identify viruses, viroids, bacteria, fungi, helminths, and/or protozoa) is measured in the biological sample. In one embodiment, the biological sample is a tissue sample that includes a tumor cell, for example, from a biopsy or formalin-fixed, paraffin-embedded (FFPE) sample. Exemplary test samples also include body fluids (e.g. blood, serum, plasma, amniotic fluid, sputum, urine, cerebrospinal fluid, lymph, tear fluid, feces, or gastric fluid), feces, tissue extracts, and culture media (e.g., a liquid in which a cell, such as a pathogen cell, has been grown). If desired, the sample is purified prior to detection using any standard method typically used for isolating a nucleic acid molecule from a biological sample. In one embodiment, a target nucleic acid of a pathogen is amplified by primer oligonucleotides to detect the presence of the nucleic acid sequence of an infectious agent in the sample. Such nucleic acid sequences may derive from pathogens including fungi, bacteria, viruses and yeast.

Target nucleic acid molecules include double-stranded and single-stranded nucleic acid molecules (e.g., DNA, RNA, and other nucleobase polymers known in the art capable of hybridizing with a nucleic acid molecule described herein). RNA molecules suitable for detection with a detectable oligonucleotide probe or detectable primer/template oligonucleotide of the invention include, but are not limited to, double-stranded and single-stranded RNA molecules that comprise a target sequence (e.g., messenger RNA, viral RNA, ribosomal RNA, transfer RNA, microRNA and microRNA precursors, and siRNAs or other RNAs described herein or known in the art). DNA molecules suitable for detection with a detectable oligonucleotide probe or primer/template oligonucleotide of the invention include, but are not limited to, double stranded DNA (e.g., genomic DNA, plasmid DNA, mitochondrial DNA, viral DNA, and synthetic double stranded DNA). Single-stranded DNA target nucleic acid molecules include, for example, viral DNA, cDNA, and synthetic single-stranded DNA, or other types of DNA known in the art. In general, a target sequence for detection is between about 30 and about 300 nucleotides in length (e.g., 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300 nucleotides). In a specific embodiment the target sequence is about 60 nucleotides in length. A target sequence for detection may also have at least about 70, 80, 90, 95, 96, 97, 98, 99, or even 100% identity to a probe sequence. Probe sequences may be longer or shorter than the target sequence. For example, a 60-nucleotide probe may hybridize to at least about 44 nucleotides of a target sequence.

In particular embodiments, a biomarker is a biomolecule (e.g., nucleic acid molecule) that is differentially present in a biological sample. For example, a biomarker is taken from a subject of one phenotypic status (e.g., having ovarian cancer) as compared with another phenotypic status (e.g., not having ovarian cancer). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease (e.g., having ovarian cancer).

### Target Capture Probes

Demonstrated herein, probe-capture next generation sequencing (NGS) was used to further validate PathoChip screen results. Genomic regions of all biomarkers as well as the viral and microbial signatures detected in ovarian cancer were pulled out using the probes that were detected positive in the PathoChip screen.

In one embodiment, the invention includes a composition comprising at least three nucleic acid probes selected from the group consisting of SEQ ID NOS: 1-94. In another embodiment, the invention includes a kit comprising at least three nucleic acid probes selected from the group consisting of SEQ ID NOS: 1-94, and instructional material for use thereof. The nucleic acid probes can be selected from between about 10 to about 30 microbes and comprise about 3 to about 5 probes per microbe.

In various embodiments, the sets of probes used herein are based on the construction of a metagenome and its use to select probes that identify target nucleic acid molecules associated with an infectious agent. As used herein “metagenome” refers to genetic material from more than one organism, e.g., in an environmental sample. The metagenome is used to select the sets of probes and/or to validate probe sets. In some embodiments, the metagenome comprises the sequences or genomes of about 20, 30, 40, 50, 60, 70, 80, 90, 100, 200, 300, 400, 500, 1000, 1500, 2000 or more organisms. In one example, the nucleic acid sequences of thousands of organisms were linked to generate a metagenome comprising 58 chromosomes.

### One Non-Limiting Example of Discrete Metagenome Probe Selection:

A. Download individual genomes, genes and partial sequences into a local database of accessions
B. Mask low complexity sequences using bioinformatic tools. In one example, low complexity sequences are masked using mdust (docdotbioperldotorg/bioperl-run/lib/Bio/Tools/Run/Mdustdothtml) followed by BLASTN 2.0MP-WashU31 identification of unique regions in viral accessions.
C. BLASTN sequence comparison of each accession against all other accessions
D. Identify specific target regions within each accession
   1. 250-300 bp regions
   2. No more than 50 contiguous nucleotides with 70% or greater sequence homology to any other accession or to the human genome
E. Supplement specific targets
   1. Identify any accessions with zero or one target region
   2. Relax stringency parameters to no more than 30 contiguous nucleotides with 50% or greater sequence homology to any other accession, but no more than 50 contiguous nucleotides with 70% or greater sequence homology to human genome
   3. Re-run target region identification on accession subset from 1.E.1.
F. Identify conserved target regions
   1. 70-300 bp regions that have 70% or greater homology with at least one other accession
   2. Remove conserved targets with 50 or more contiguous nucleotides with 70% or greater sequence homology to human genome
G. Choose probes
   1. Run Agilent array CGH probe selection algorithm on specific and conserved target regions
   2. Rank probes by Agilent design score
   3. Select 1-3 highest ranking probes from 1-5 specific target regions in each accession
   4. Select 1-3 highest ranking probes from each conserved target region

Concatenated metagenome probe selection
A. Download individual genomes, genes and partial sequences into a local database of accessions
B. Compile all accessions into a single concatenated metagenome to facilitate use of genomics bioinformatics tools
   1. Place 100 nonspecific nucleotides (“N”) as spacers between each accession
   2. Join accessions and spacers into chromosomes of 6-10 million bases
C. Run Agilent array CGH probe selection algorithm for specificity within the metagenome
D. Filter probes for specificity against human, mouse, and/or other mammalian genomes
E. Choose specific probes
   1. Rank probes by Agilent design score
   2. Select 10-20 highest ranking probes from each accession
   3. Require at least 100 bp separation between probes
F. Choose conserved probes
   1. Identify conserved regions as in 1.F.
   2. Select 5-10 highest ranking probes from each conserved region
   3. Require at least 100 bp separation between probes
G. Empirical probe selection
   1. Manufacture microarrays containing all specific and conserved probes
   2. Hybridize microarrays to labeled human DNA
   3. Select 5-10 specific probes from each accession with lowest cross-hybridization signal
   4. Select 3-5 conserved probes from each conserved regions with lowest cross-hybridization signal

### Sample Preparation

The invention provides a means for analyzing multiple types of nucleic acids present in a sample, including DNA and RNA. In various embodiments, sample preparation involves extracting a mixture of nucleic acid molecules (e.g., DNA and RNA). In other embodiments, sample preparation involves extracting a mixture of nucleic acids from multiple organisms, cell types, infectious agents, or any combination thereof. In one embodiment, sample preparation involves the workflow below.
A. Fragment genomic DNA
B. Convert total RNA to first strand cDNA by random-primed reverse transcriptase
C. Label genomic DNA with biotin or fluorescent dye by chemical or enzymatic incorporation
D. Label cDNA with biotin or fluorescent dye by chemical or enzymatic incorporation
E. Label a mixture of genomic DNA and cDNA in the same chemical or enzymatic reaction
F. Mix C+D and co-hybridize to microarray of probes
G. Hybridize E to microarray of probes
H. Amplify targeted genomic DNA
   1. Use whole-genome amplification (GE GenomiPhi, Sigma WGA, NuGEN Ovation DNA) to non-specifically amplify genomic DNA
   2. Use amplified products as input for steps C, or E.
I. Amplify targeted total RNA
   1. Use whole-transcriptome amplification (Sigma WTA, Ambion in vitro transcription, NuGEN Ovation RNA) to non-specifically amplify total RNA
   2. Use amplified products as input.

The samples are hybridized to the microarray (e.g., PathoChip), and the microarrays are washed at various stringencies. Microarrays are scanned for detection of fluorescence. Background correction and inter-array normalization algorithms are applied. Detection thresholds are applied. The results are analyzed for statistical significance.

### Nucleic Acid Amplification

Target nucleic acid sequences are optionally amplified before being detected. The term “amplified” defines the process of making multiple copies of the nucleic acid from a single or lower copy number of nucleic acid sequence molecule. The amplification of nucleic acid sequences is carried out in vitro by biochemical processes known to those of skill in the art. Prior to or concurrent with identification, the viral sample may be amplified by a variety of mechanisms, some of which may employ PCR. For example, primers for PCR may be designed to amplify regions of the sequence. For RNA viruses a first reverse transcriptase step may be used to generate double stranded DNA from the single stranded RNA. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Manila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159, 4,965,188, and 5,333,675. The sample may be amplified on the array. See, for example, U.S. Pat. No. 6,300,070 and U.S. Ser. No. 09/513,300.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed PCR (CP-PCR) (U.S. Pat. No. 4,437,975), arbitrarily primed PCR (AP-PCR) (U.S. Pat. Nos. 5,413,909, 5,861,245) and nucleic acid based sequence amplification (NABSA) (see, U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317.

Additional methods of sample preparation and techniques for reducing the complexity of a nucleic acid sample are described in Dong et al., Genome Research 11, 1418 (2001), in U.S. Pat. Nos. 6,361,947, 6,391,592 and U.S. Ser. Nos. 09/916,135, 09/920,491 (US Patent Application Publication 20030096235), Ser. No. 09/910,292 (US Patent Application Publication 20030082543), and Ser. No. 10/013,598.

### Detection of Biomarkers

The biomarkers of this invention can be detected by any suitable method. The methods described herein can be used individually or in combination for a more accurate detection of the biomarkers. Methods for conducting polynucleotide hybridization assays have been developed in the art. Hybridization assay procedures and conditions will vary depending on the application and are selected in accordance with the general binding methods known including those referred to in: Sambrook and Russell, Molecular Cloning: A Laboratory Manual (3^{rd} Ed. Cold Spring Harbor, N.Y., 2001); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623. A data analysis algorithm (E-predict) for interpreting the hybridization results from an array is publicly available (see Urisman, 2005, Genome Biol 6:R78).

In one embodiment, the hybridized nucleic acids are detected by detecting one or more labels attached to, or incorporated within, the sample nucleic acids. The labels may be attached or incorporated by any of a number of means well known to those of skill in the art. In one embodiment, the label is simultaneously incorporated during the amplification step in the preparation of the sample nucleic acids. Thus, for example, PCR with labeled primers or labeled nucleotides will provide a labeled amplification product. In another embodiment, transcription amplification, as described above, using a labeled nucleotide (e.g. fluorescein-labeled UTP and/or CTP) incorporates a label into the transcribed nucleic acids. In another embodiment PCR amplification products are fragmented and labeled by terminal deoxytransferase and labeled dNTPs. Alternatively, a label may be added directly to the original nucleic acid sample (e.g., mRNA, polyA mRNA, cDNA, etc.) or to the amplification product after the amplification is completed. Means of attaching labels to nucleic acids are well known to those of skill in the art and include, for example, nick translation or end-labeling (e.g. with a labeled RNA) by kinasing the nucleic acid and subsequent attachment (ligation) of a nucleic acid linker joining the sample nucleic acid to a label (e.g., a fluorophore). In another embodiment label is added to the end of fragments using terminal deoxytransferase.

Detectable labels suitable for use in the present invention include any composition detectable by spectroscopic, photochemical, biochemical, immunochemical, electrical, optical or chemical means. Useful labels in the present invention include, but are not limited to: biotin for staining with labeled streptavidin conjugate; anti-biotin antibodies, magnetic beads (e.g., Dynabeads™); fluorescent dyes (e.g., Cy3, Cy5, fluorescein, texas red, rhodamine, green fluorescent protein, and the like); radiolabels (e.g., ³H, ¹²⁵I, ³⁵S, ⁴C or ³²P); phosphorescent labels; enzymes (e.g., horse radish peroxidase, alkaline phosphatase and others commonly used in an ELISA); and colorimetric labels such as colloidal gold or colored glass or plastic (e.g., polystyrene, polypropylene, latex, etc.) beads. Patents teaching the use of such labels include U.S. Pat. Nos. 3,817,837, 3,850,752, 3,939,350, 3,996,345, 4,277,437, 4,275,149 and 4,366,241.

Means of detecting such labels are well known to those of skill in the art. Thus, for example, radiolabels may be detected using photographic film or scintillation counters; fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Ser. Nos. 10/389,194, 60/493,495 and in PCT Application PCT/US99/06097 (published as WO99/47964).

### Detection by Microarray

In certain aspects of the invention, a sample is analyzed by means of a microarray. The nucleic acid molecules of the invention are useful as hybridizable array elements in a microarray. Microarrays generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. U.S. Pat. Nos. 5,800,992 and 6,040,138 describe methods for making arrays of nucleic acid probes that can be used to detect the presence of a nucleic acid containing a specific nucleotide sequence. Methods of forming high-density arrays of nucleic acids, peptides and other polymer sequences with a minimal number of synthetic steps are known. The nucleic acid array can be synthesized on a solid substrate by a variety of methods, including, but not limited to, light-directed chemical coupling, and mechanically directed coupling. For additional descriptions and methods relating to resequencing arrays see U.S. patent application Ser. Nos. 10/658,879, 60/417,190, 09/381,480, 60/409,396, and U.S. Pat. Nos. 5,861,242, 6,027,880, 5,837,832, 6,723,503.

By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507). For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

One embodiment of the invention includes a microarray comprising at least three nucleic acid probes selected from the group consisting of SEQ ID NOS: 1-94. The nucleic acid probes can be selected from about 10 to about 30 microbes and comprise about 3 to about 5 probes per microbe. In another embodiment, the microarray comprises at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria. The microarray can be a biochip, or on a glass slide, bead, or paper.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure. Exemplary nucleic acid molecules useful in the invention include polynucleotides that specifically bind nucleic acid biomarkers to one or more pathogenic organisms, and fragments thereof.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy); or a cell or population of cells isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection systems for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In certain embodiments, a scanner is used to determine the levels and patterns of fluorescence.

### Diagnostic Assays

The present invention provides a number of diagnostic assays that are useful for the identification or characterization of a disease or disorder (e.g., ovarian cancer), or a propensity to develop such a condition. In one embodiment, ovarian cancer is characterized by quantifying the level of one or more biomarkers from one or more pathogenic organisms, including viruses, viroids, bacteria, fungi, helminths, and protozoa. While the examples provided herein describe specific methods of detecting levels of these markers, the skilled artisan appreciates that the invention is not limited to such methods. Marker levels are quantifiable by any standard method, such methods include, but are not limited to real-time PCR, Southern blot, PCR, and/or mass spectroscopy.

The level of any two or more of the markers described herein defines the marker profile of a disease, disorder, or condition. The level of marker is compared to a reference. In one embodiment, the reference is the level of marker present in a control sample obtained from a patient that does not have ovarian cancer. In another embodiment, the reference is a healthy tissue or cell (i.e., that is negative for ovarian cancer). In another embodiment, the reference is a baseline level of marker present in a biologic sample derived from a patient prior to, during, or after treatment for ovarian cancer. In yet another embodiment, the reference is a standardized curve. The level of any one or more of the markers described herein (e.g., a combination of viral, bacterial, fungal, helminth, and/or protozoan biomarkers) is used, alone or in combination with other standard methods, to characterize the disease, disorder, or condition (e.g., ovarian cancer).

In certain embodiments, one or more organisms described herein may be isolated or extracted from a sample using a capture reagent (e.g., an antibody) and/or detected using ELISA. In a particular embodiment, reagents for capturing the pathogenic organism include Streptavidin bound magnetic beads and biotin labeled probes. Such techniques can be further used to obtain nucleic acids pathogenic organism detection using nucleic acid based probes or for direct sequencing (e.g., MiSeq; Illumina).

### Kits

The invention provides kits for the detection of a biomarker, which is indicative of the presence of one or more biological sequences or agents associated with ovarian cancer. The kits may be used for detecting the presence of multiple biological agents associated with ovarian cancer. The kits may be used for the diagnosis or detection of ovarian cancer. In some embodiments, the kit comprises a panel or collection of probes to nucleic acid biomarkers (e.g., PathoChip) delineated herein as specific for detection of ovarian cancer. In additional or alternative embodiments, the kit comprises an antibody specific for a pathogenic organism associated with ovarian cancer. Such antibodies may be used for ELISA detection or for extraction of a pathogenic organism associated with ovarian cancer (e.g., a biotin labeled antibody in conjunction with Streptavidin bound magnetic beads).

In some embodiments, the kit comprises one or more sterile containers which contain the panel of probes, nucleic acid biomarkers, or microarray chip. Such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

The instructions will generally include information about the use of the composition for the detection or diagnosis of ovarian cancer. In other embodiments, the instructions include at least one of the following: description of the therapeutic agent; dosage schedule and administration for treatment or prevention of ovarian cancer or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

One embodiment of the invention is a kit comprising at least three nucleic acid probes selected from the group consisting of SEQ ID NOS: 1-94. The kit can include probes from about 10-30 organisms with about 3-5 probes per organism. Another embodiment of the invention is a kit comprising a microarray with at least three nucleic acid probes selected from the group consisting of SEQ ID NOS: 1-94. In another embodiment, the kit comprises a microarray comprising at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptomphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed herein.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the exemplary embodiments of the present invention, and are not to be construed as limiting in any way the remainder of the disclosure.

The materials and methods employed in these experiments are now described.

Study Samples:

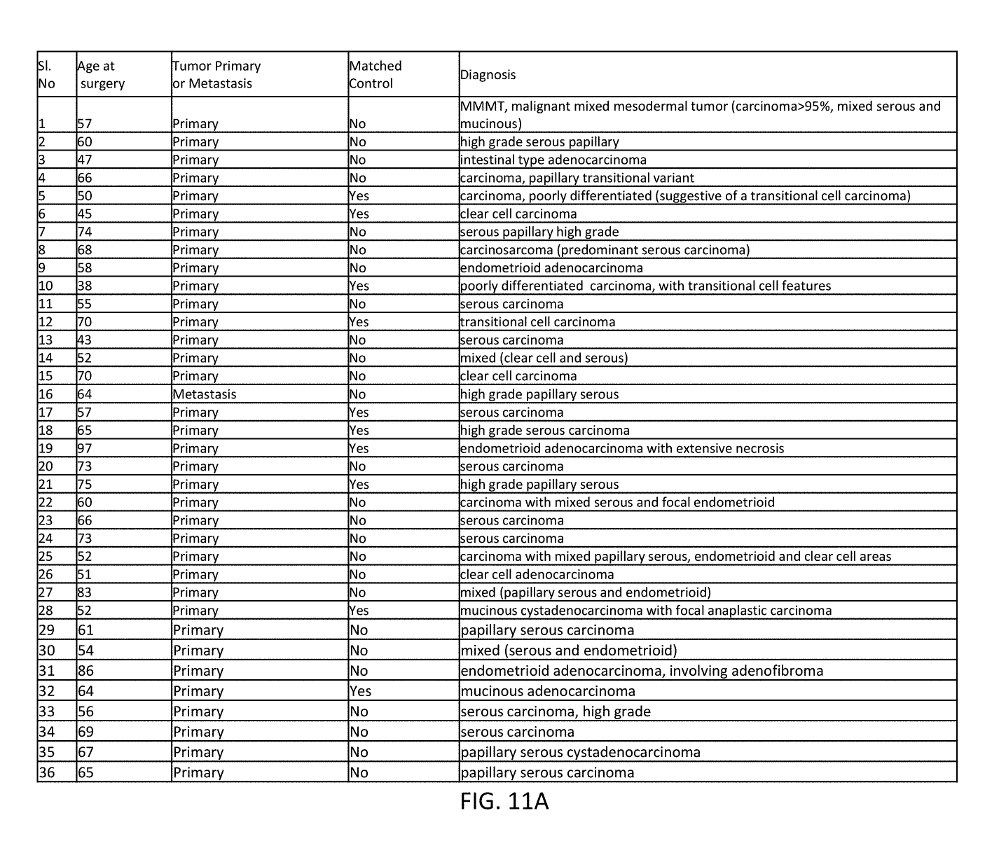

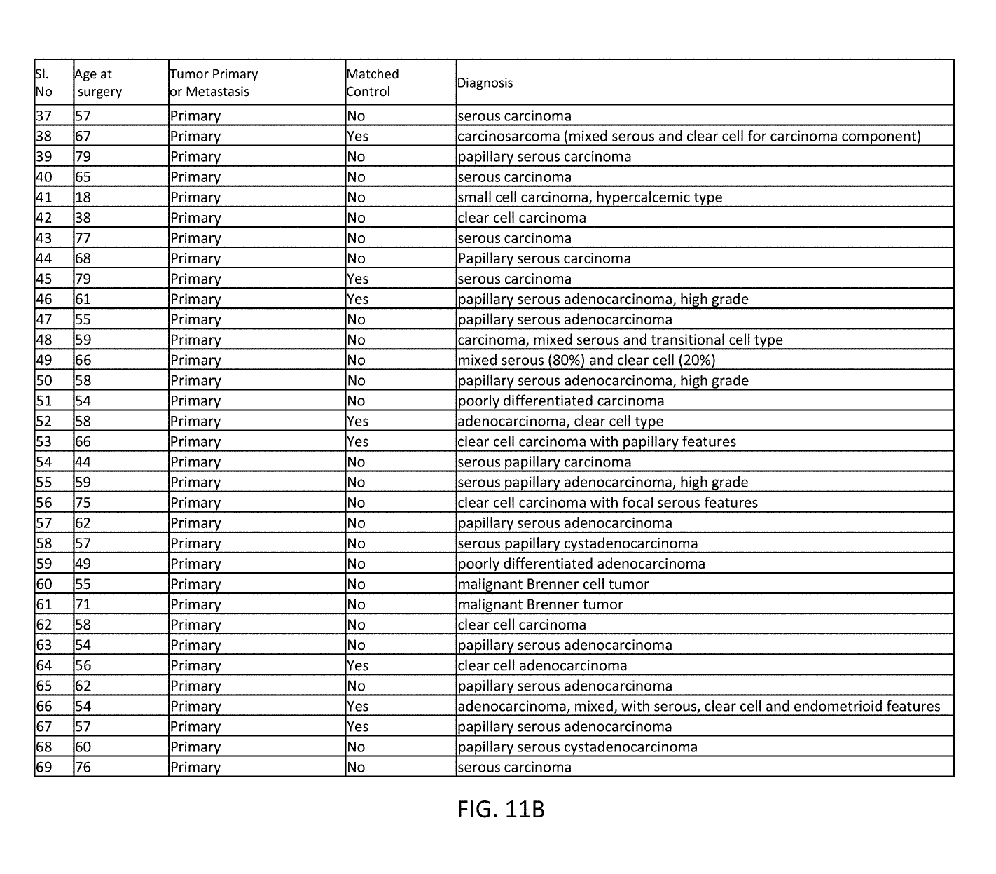

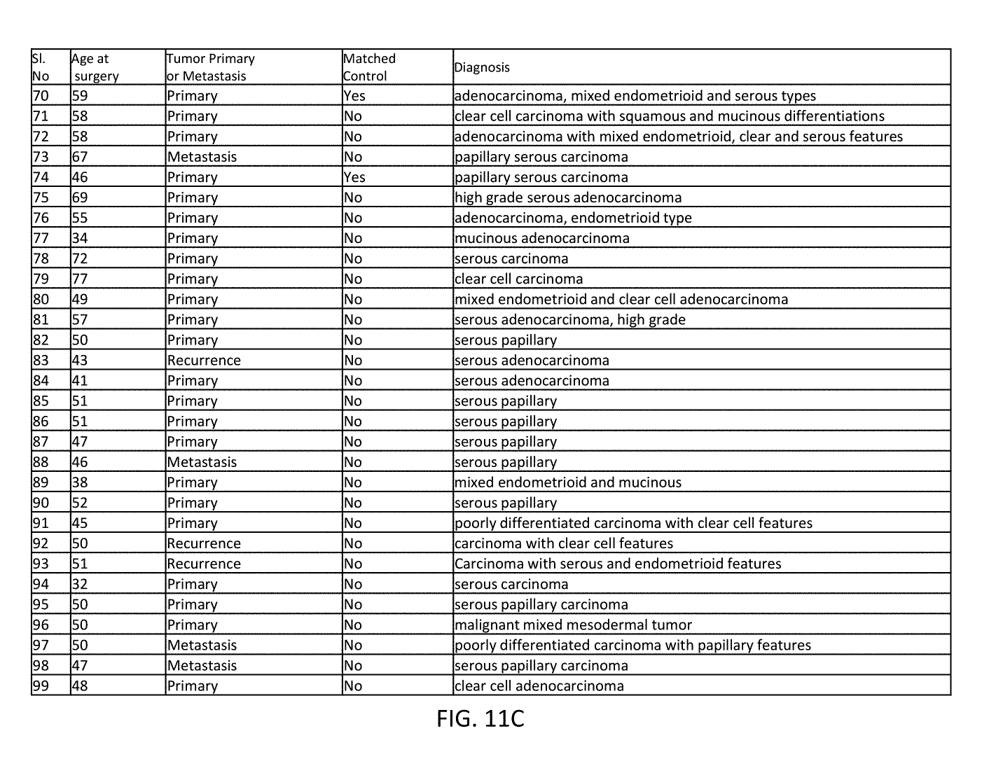

The computerized records at the a) Tumor Tissue and Biospecimen Bank and b) the clinical archives of the Department of Pathology and Laboratory Medicine at the University of Pennsylvania were searched and a total of 99 primary and recurrent or metastatic tumors of ovarian origin were identified (FIGS. 11A-11C). Both the metastatic or recurrent tumor were still of ovarian origin. Histology of the cases evaluated included malignant surface epithelial tumors (serous, endometrioid, mucinous, clear cell, transitional cell, mixed types and carcinosarcoma) and 1 case of small cell carcinoma, hypercalcemic type. The matched control tissues were non-tumor ovarian tissue from ipsilateral or contralateral ovary from 20 ovarian cancer patients (FIGS. 11A-11C). The non-matched control benign tissues were from prophylactic oophorectomy surgery in women with BRCA mutations.

The original H&E slides were reviewed and one representative formalin-fixed, paraffin-embedded tissue block was chosen per case and cut. Tumors needing macro-dissection were received in the form of 10 pm sections on glass slides with marked guiding H&E slides, while tumors that did not require macro-dissection were received as 10 μm paraffin rolls.

PathoChip Design, Sample Preparation and Microarray Processing:

The PathoChip Array design has been previously described in detail (Banerjee et al. Sci Rep. 2015; 5:15162; Baldwin et al. MBio. 2014; 5: e01714-14). Briefly, the probes were generated in silico from a metagenome of 58 chromosomes comprising the genomes of all known viruses as well as known human bacterial, parasitic and fungal pathogens (Baldwin et al. MBio. 2014; 5: e01714-14). PathoChip comprises 60,000 probe sets manufactured as SurePrint glass slide microarrays (Agilent Technologies Inc.), containing 8 replicate arrays per slide. Each probe is a 60-nt DNA oligomer that targets multiple genomic regions of the viruses and higher pathogens.

PathoChip screening was done using both DNA and RNA extracted from formalin-fixed paraffin-embedded (FFPE) tumor tissues as described previously (Banerjee et al. Sci Rep. 2015; 5:15162; Baldwin et al. MBio. 2014; 5: e01714-14). 99 de-identified FFPE samples of invasive epithelial malignant tumors of ovarian origin were received as 10 pm sections on non-charged glass slides from the Abramson Cancer Center Tumor Tissue and Biosample Core. Additionally, 20 matched and 20 non-matched control samples were provided as paraffin rolls. Matched controls were obtained from the adjacent non-cancerous ovarian tissue of the same patient from which the cancer tissues are obtained, non-matched controls were the ovarian tissues obtained from non-cancerous individuals. DNA and RNA were extracted in parallel from 5 rolls or mounted sections of each FFPE sample. The quality of the extracted nucleic acids was determined by agarose gel electrophoresis and the A260/280 ratio. The extracted RNA and DNA samples were subjected to whole transcriptome amplification (WTA) as previously described (Banerjee et al. Sci Rep. 2015; 5:15162; Baldwin et al. MBio. 2014; 5: e01714-14). The WTA products were analyzed by agarose gel electrophoresis. Human reference RNA and DNA were also extracted from the human B cell line, BJAB and were used for WTA as previously described (Banerjee et al. Sci Rep. 2015; 5:15162; Baldwin et al. MBio. 2014; 5: e01714-14). The WTA products were purified, (PCR purification kit, Qiagen, Germantown, Md., USA); the WTA products from the ovarian cancers were labelled with Cy3 and those from the human reference DNA were labelled with Cy5 (SureTag labeling kit, Agilent Technologies, Santa Clara, Calif.). The labelled DNAs were purified and hybridized to the PathoChip as described previously (Banerjee et al. Sci Rep. 2015; 5:15162; Baldwin et al. MBio. 2014; 5: e01714-14). Post-hybridization, the slides were washed, scanned and visualized using an Agilent SureScan G4900DA array scanner. Microarray Data Extraction and Statistical analysis: The microarray data extraction and analyses have been described previously (Banerjee et al. Sci Rep. 2015; 5:15162; Baldwin et al. MBio. 2014; 5: e01714-14). The raw data from the microarray images were extracted using Agilent Feature Extraction software; Apart from the previously described method, the R program for normalization and data analyses was used. Scale factor was calculated using the signals of green and red channels for human probes to calculate scale factor. Scale factors are the sum of green/sum of red signal ratios of human probes. Scale factors were then used to obtain normalized signals for all other probes. For all probes except human probes, normalized signal is log 2 transformed of green signals/scale factors modified red signals (log 2 g−log 2 scale factor*r). On the normalized signals, t-test is applied to select probes significantly present in cancer samples by comparing cancer samples versus controls (un-matched and matched controls) and to select probes significantly present in un-matched or matched controls versus the cancer samples. The significance cutoff was log 2 fold change >0.5 and adjusted p-value<0.05. The adjusted p-values were obtained for multiple corrections by using the Benjamini-Hochberg procedure (Benjamini and Hochberg, J Royal Statist Society. 1995; Series B. 57 289-300). No significant ones were detected in control under this adjusted p-value cutoff. Presented are the top ones in control with nominal p-value<0.05 without any multiple comparison correction, in order to have a comparison with the significant ones present in cancer samples. Prevalence was calculated based on the detection of the signatures in the cancer and the control samples as percentage.

The cancer samples were also subjected to hierarchical clustering, based on the detection of microbial signatures in the samples, using the R program (Euclidean distance, complete linkage, non-adjusted values) (R Core Team. R: A language and environment for statistical computing. R Foundation for Statistical Computing, Vienna, Austria. 2015; Kolde R and pheatmap: Pretty Heatmaps. R package version 1.0.2. 2015). Additional topological-based data analyses were conducted using the Ayasdi software (Ayasdi, Inc.), (using Euclidean (L2) metric, and L-infinity centrality lense), where statistical significance between different groups was determined using the two-sided t-test.

Probe Capture and Next Generation Sequencing:

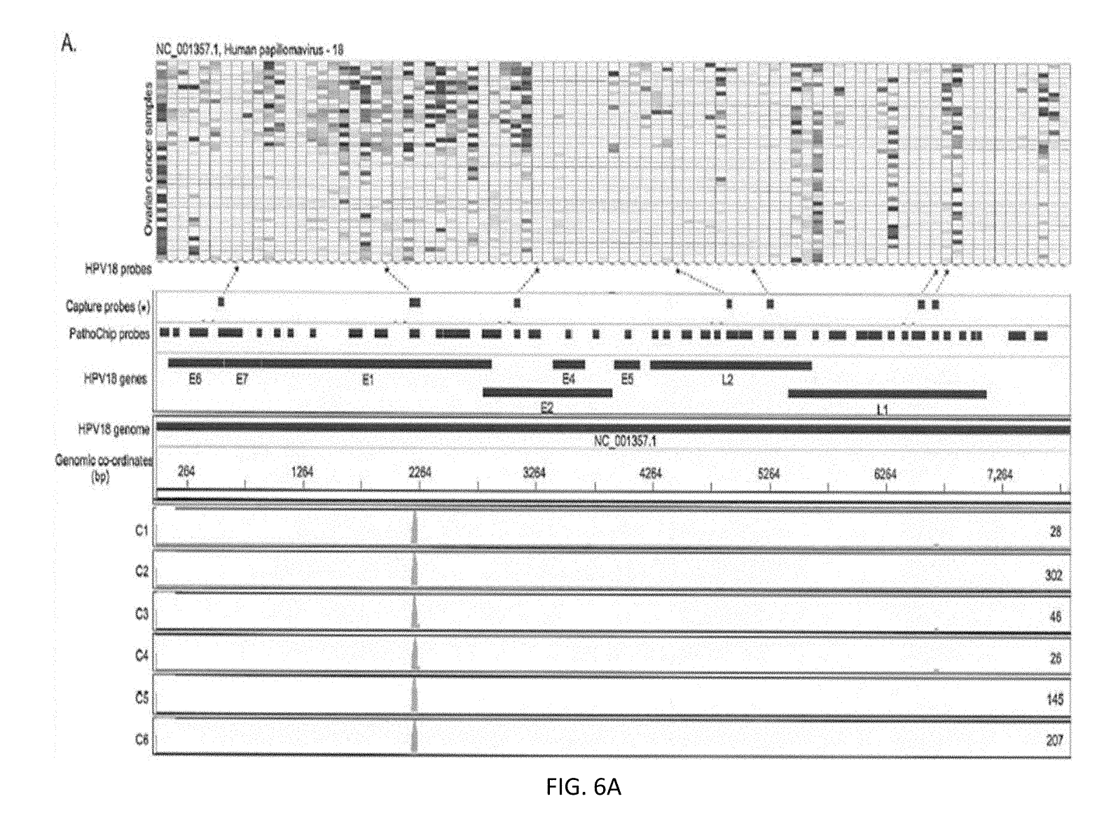

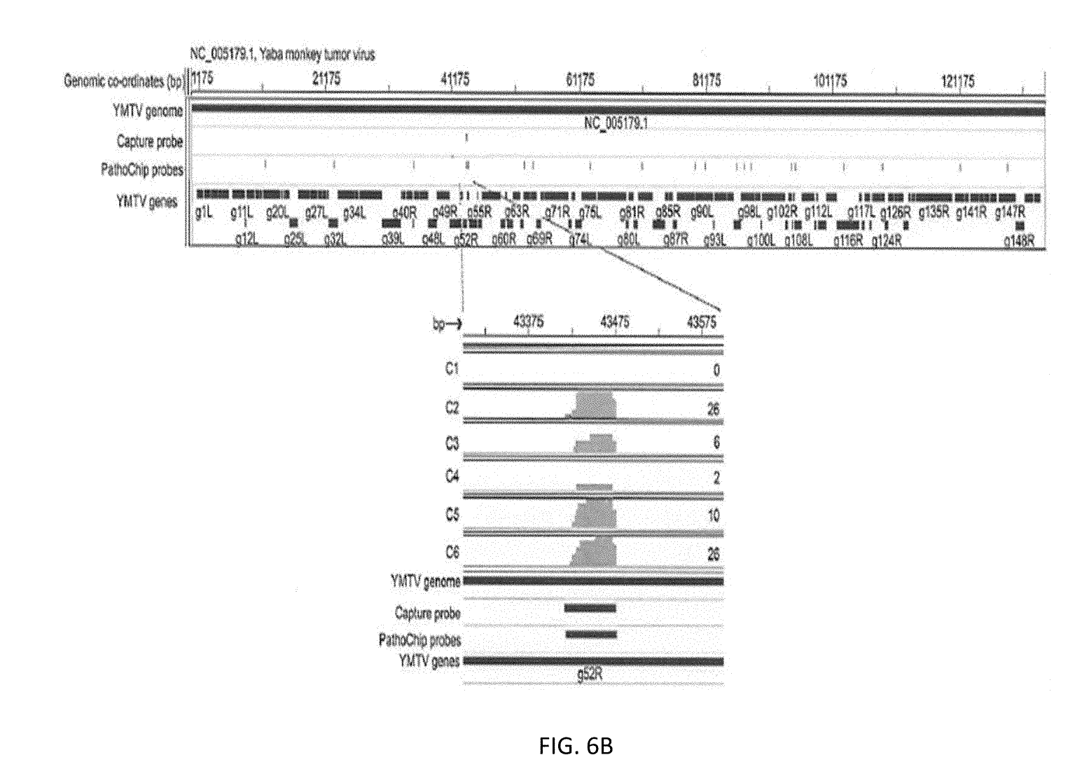

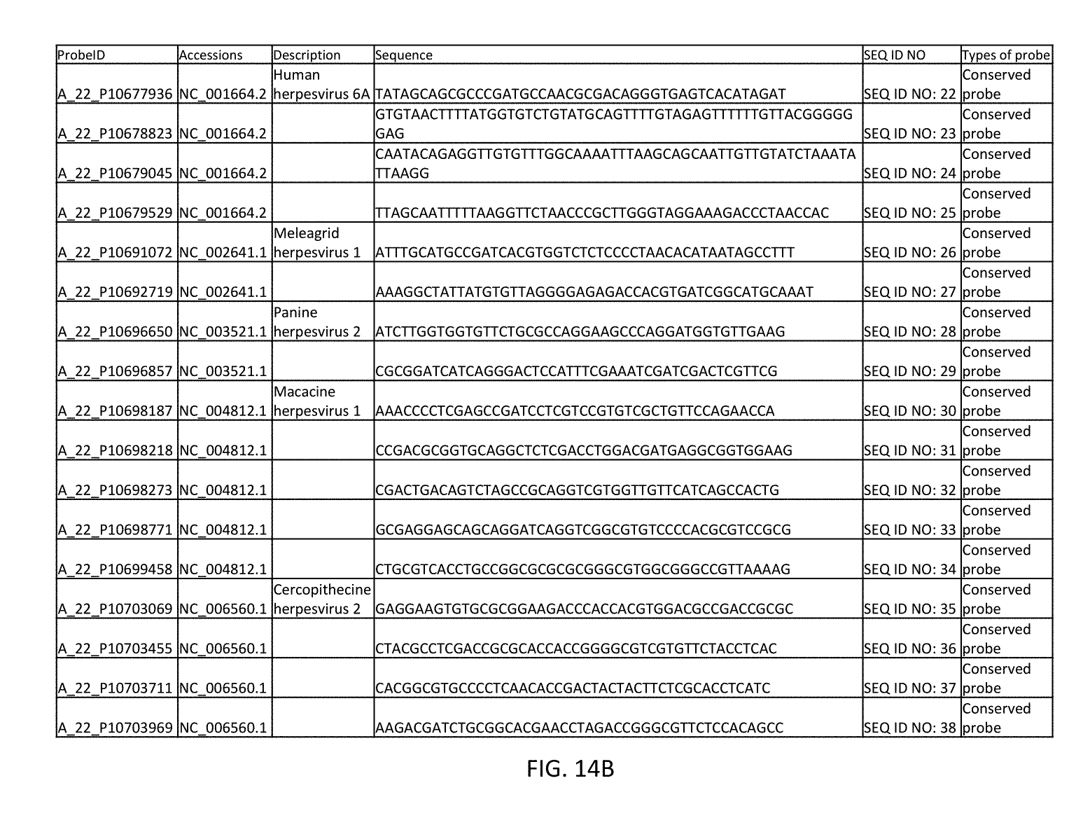

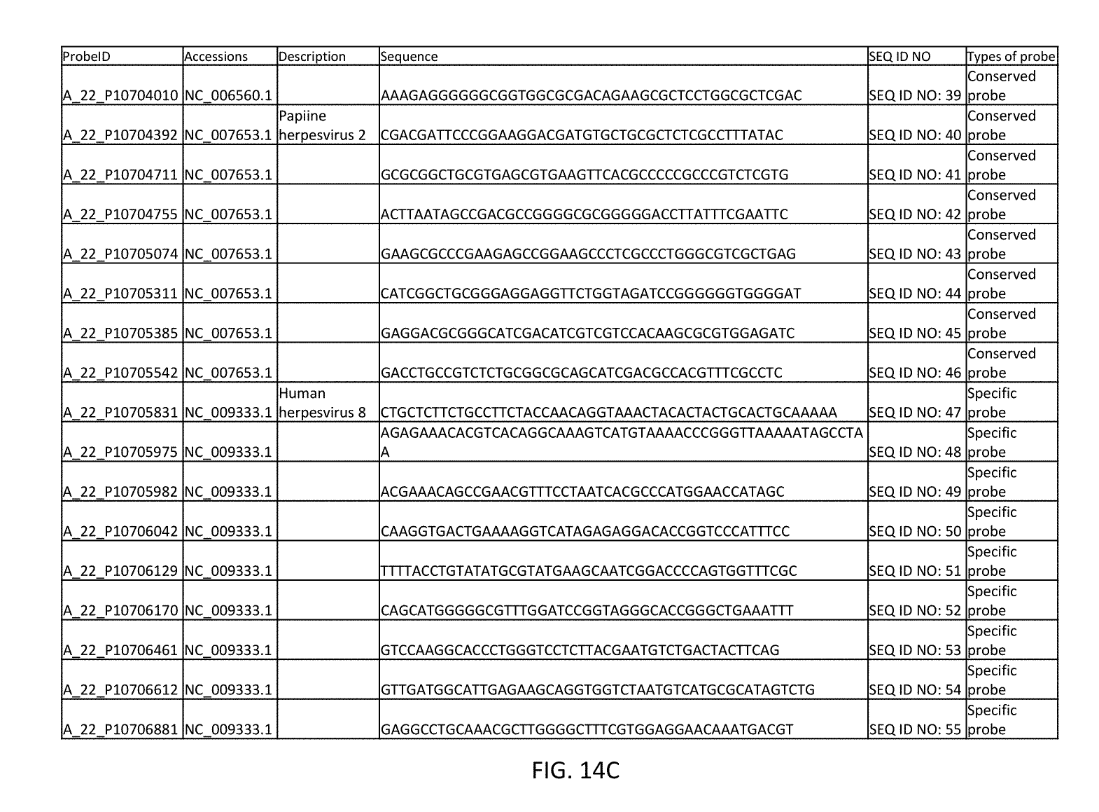

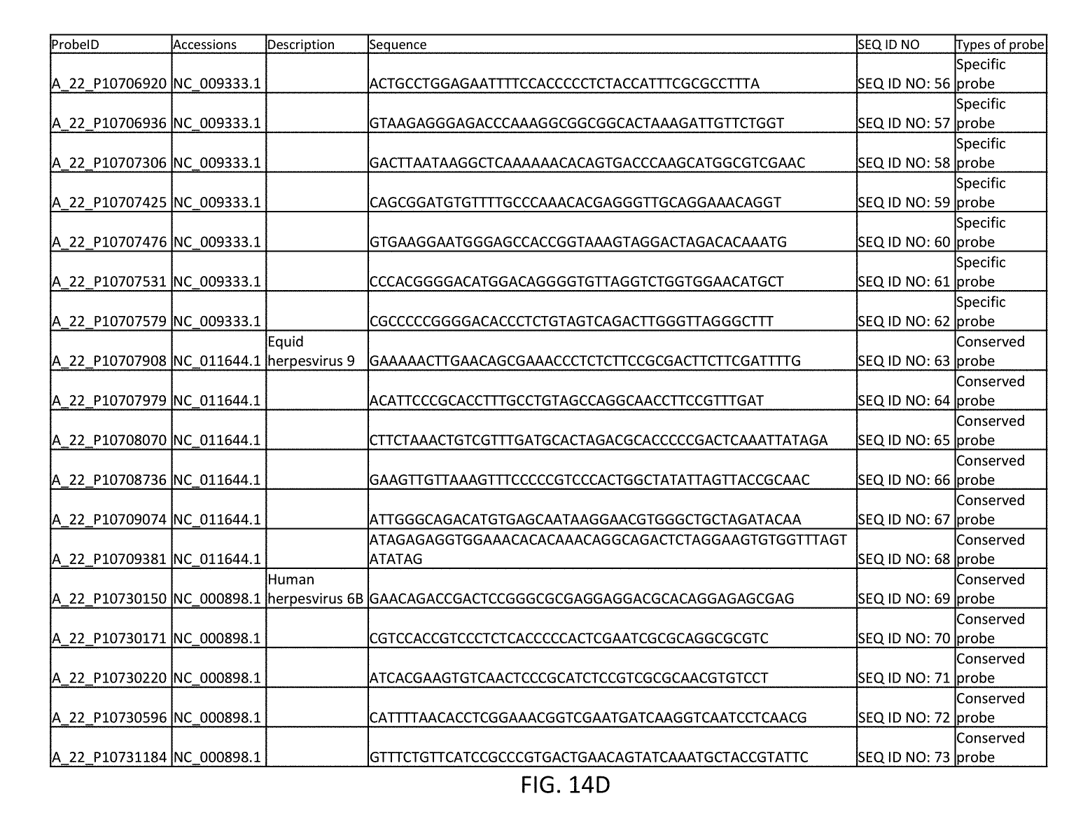

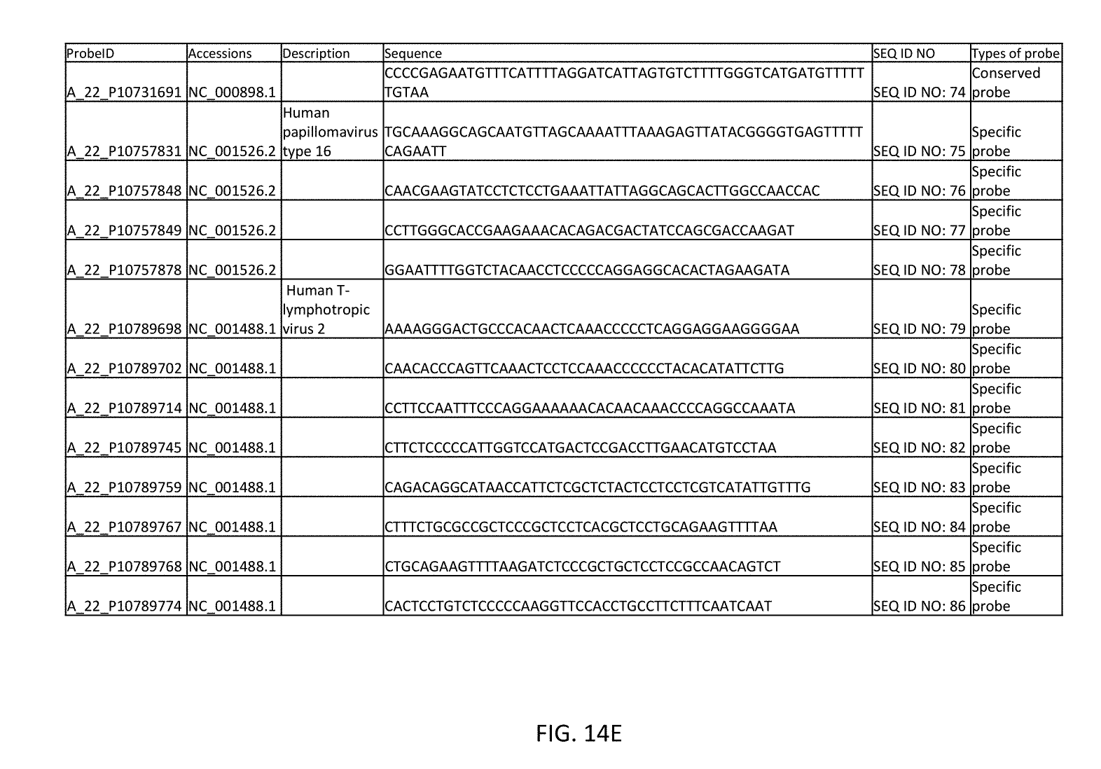

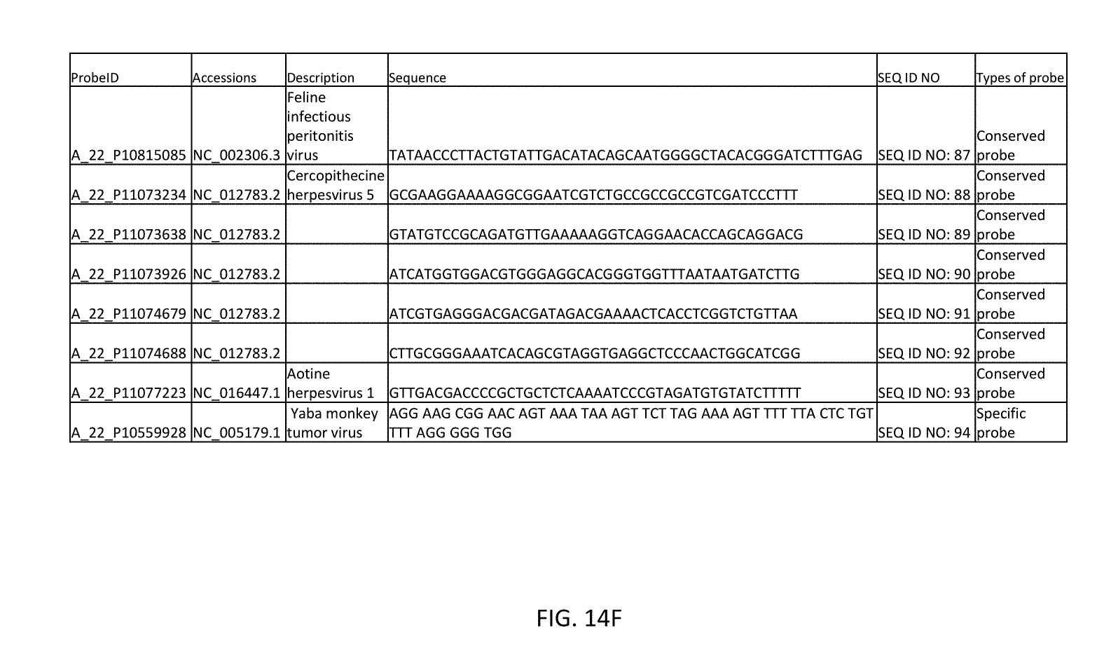

Probe Capture method has been previously described (Banerjee et al. Sci Rep. 2015; 5:15162; Baldwin et al. MBio. 2014; 5: e01714-14). Briefly, selected PathoChip probes that identified microbial signatures in the ovarian cancer samples were made as biotinylated derivatives and used to capture the microbial target nucleic acid from pooled WTA products from the ovarian cancer samples. Hybridization was followed by capturing the targeted sequences using Streptavidin coated magnetic beads. The libraries of the targets were generated for NGS using Nextera XT sample preparation kit (Illumina, San Diego, Calif., USA). Six libraries were generated, ovl-6. The selected probes used for the target capture are listed in (FIGS. 14A-14F). The libraries were submitted to the Washington University Genome Technology Access Center (St. Louis, Mo.) for quality control measurements, library pooling, and sequencing using an Illumina MiSeq instrument with paired-end 250-nt reads. Adapters and low-quality fragments of raw reads were first removed using the Trim Galore software. The processed reads were then aligned to the PathoChip metagenome and the human genome using Genomic Short-read Nucleotide Alignment Program (GSNAP) (Thorvaldsdottir et al., Brief Bioinform. 2013; 14:178-192; Wu et al., Bioinformatics. 2010; 26:873-881) with default parameters. Alignment featureCounts (Liao et al. Bioinformatics. 30:923-930) was employed to count the number of reads aligned to each of the capture probe regions, and visualized in IGV (FIGS. 6A-6B).

Virus Fusion Identification:

Prior to fusion detection, raw reads were trimmed in order to remove adapters and low-quality fragments by Trim Galore software (www dot bioinformatics dot babraham dot ac dot uk/proiects/trim galore/). Virus-Clip (Ho et al., Oncotarget. 6:20959-20963) was used to identify the virus fusion sites in the human genome. Specifically, the virus genome was used as the primary read alignment target, and first aligned the reads to the PathoChip metagenome. Some of the mapped reads contained soft-clipped segments, which were then extracted from the alignment (potentially containing sequences of pathogen-integrated human loci) and mapped to the human genome. Using this mapping information, the exact human and pathogen integration breakpoints could be pinpointed at single-base resolution. All the integration sites were then automatically annotated with the affected human genes and their corresponding gene co-ordinates from the human genome maps.

The affected host genes at or near the viral genomic integration sites were analysed by Ingenuity Pathway software to determine if there were any significant association with cancer (Kramer et al., Bioinformatics. 2014; 30:523-530).

The results of the experiments are now described.

### Example 1: Microbial Signatures Uniquely Associated with Ovarian Cancer

The PathoChip technology was used to screen ovarian cancer samples, as well as matched and non-matched controls. To establish the microbiome signatures the average hybridization signal for each probe in the cancer samples versus the controls were compared. Those probes that detected significant hybridization signals in the cancer samples (p-value<0.05, log fold change in hybridization signal>log 1), were considered. Additionally, the percent prevalence of the specific microbial signatures in the cancer samples was calculated. These data indicated how prevalent a significant virus or microorganism signature was in the cancer samples regardless of the hybridization intensity. Similarly, microbiome signatures were also detected in the matched and non-matched control samples versus the ovarian cancer samples. The signature of non-matched controls was quite distinct while there was more similarity between the tumor tissue and the matched controls. However, there were district viral and microbial signatures in the tumor-specific signature.

### Example 2: Viral Signatures Associated with Ovarian Cancer

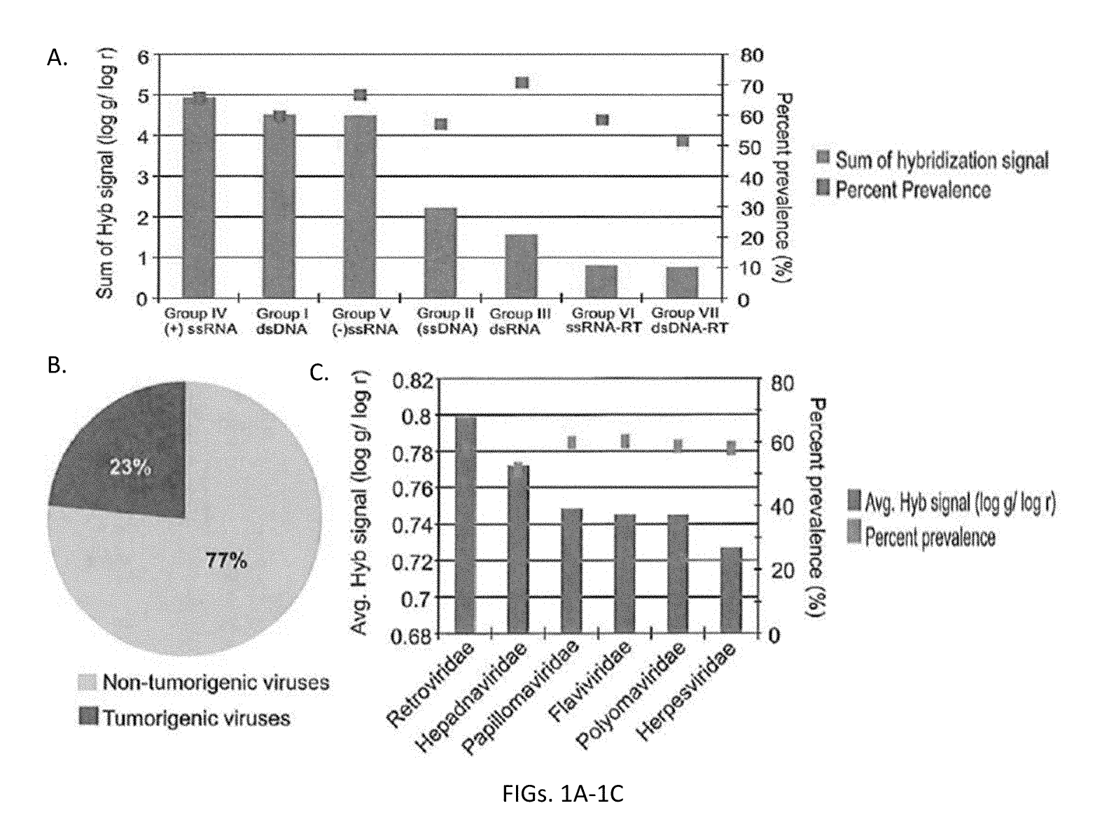

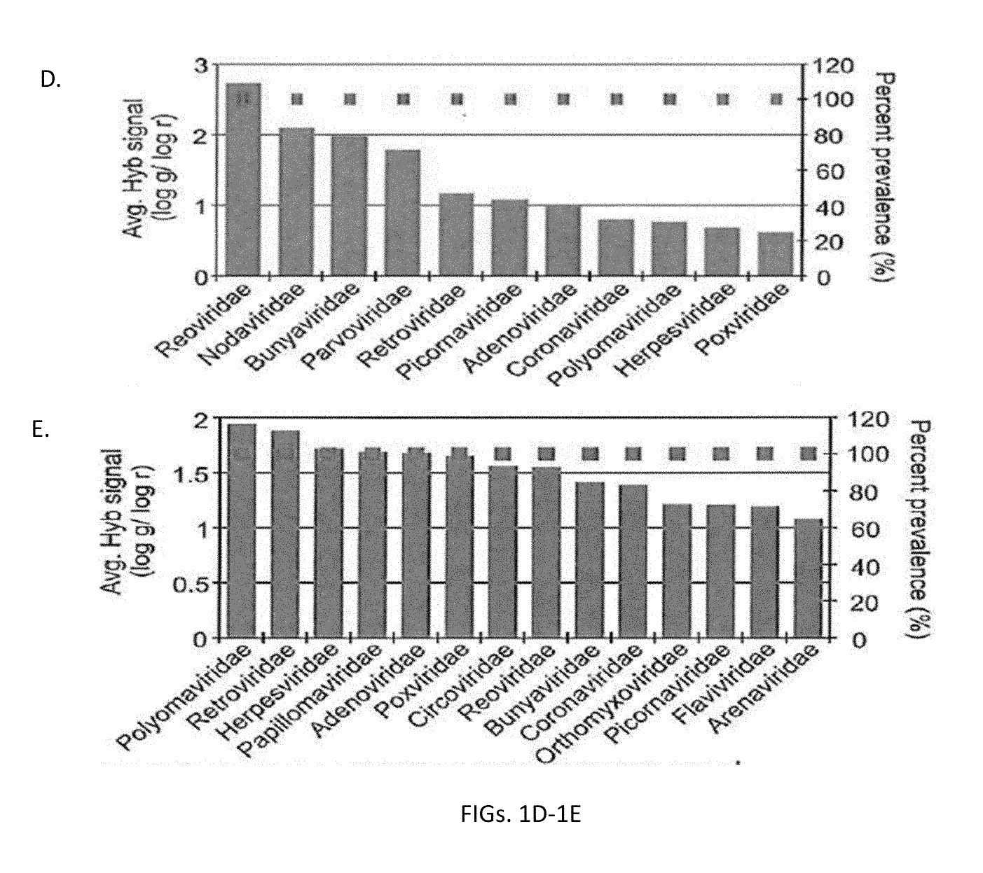

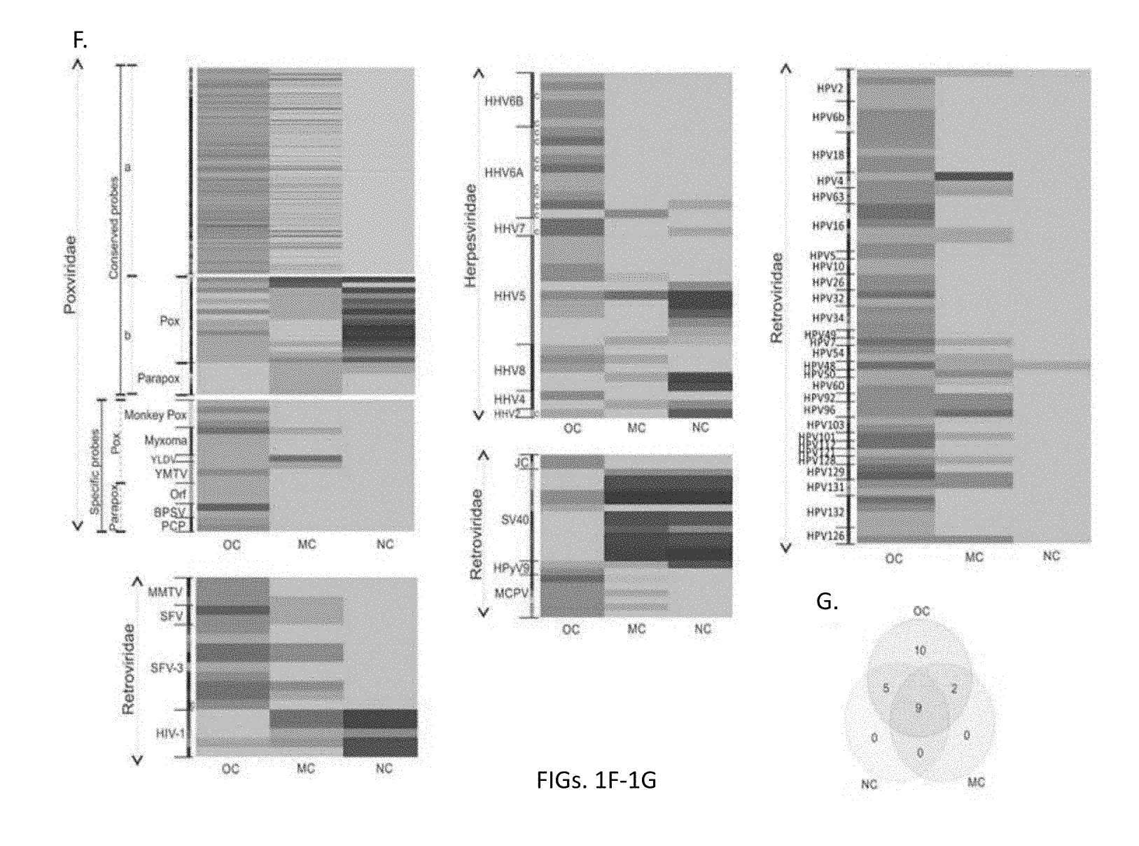

Initial analyses focused on viral signatures associated with ovarian cancer compared to matched and non-matched control samples (FIGS. 1A-1G). These viral signatures detected in the ovarian cancer and control samples are shown according to their decreasing hybridization signal along with their prevalence (FIGS. 1A-1E). The predominant signatures detected in the ovarian cancers were positive sense single stranded RNA viruses, double stranded DNA viruses and negative sense single stranded RNA viruses (FIG. 1A). Among the signatures for viral families detected, 23% were identified as tumorigenic viruses (FIG. 1B), and were prevalent on average, in more than 50% of the cancer samples screened. Signatures of Retroviridae showed the highest hybridization signal, followed by that of Hepadnaviridae, Papillomaviridae, Flaviviridae, Polyomaviridae and Herpesviridae (FIG. 1C). Notably, Papillomaviridae family members have previously been shown to be associated with ovarian cancer. Interestingly, papillomaviral signatures were found in the cancer samples and in the non-matched controls, but not at significant levels in the matched controls. The papilloma signatures in the ovarian cancer samples screened included not only HPV16 and 18 but also other HPVs (HPV-2, 4, 5, 6b, 7, 10, 32, 48, 49, 50, 60, 54, 92, 96, 101, 128, 129, 131, 132) (FIG. 1F). However the HPV signatures in matched controls that showed significant high hybridization signal intensity over those in cancer samples, were HPV 41, 88, 53 and 103 (FIG. 1F). An abundance of other viral signatures were also found in the ovarian cancer samples, (FIGS. 8A-8B, FIG. 1F, FIG. 9), including Herpesviridae (HHV4, HHV8, HHV5, HHV6a, HHV 6b), Poxviridae (both pox and parapoxvirus), Polyomaviridae (Merkel cell polyomavirus, JC polyomavirus, Simian virus 40), Retroviridae (Simian foamy virus, Mouse mammary tumor virus).

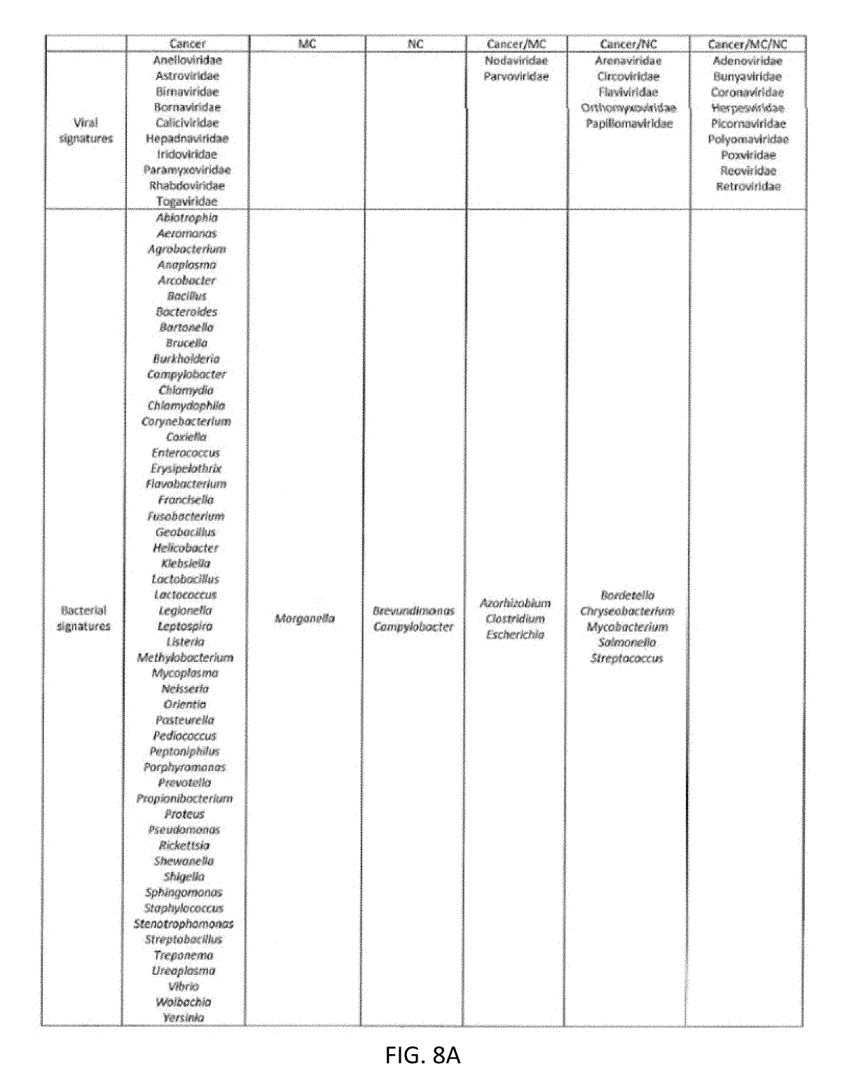

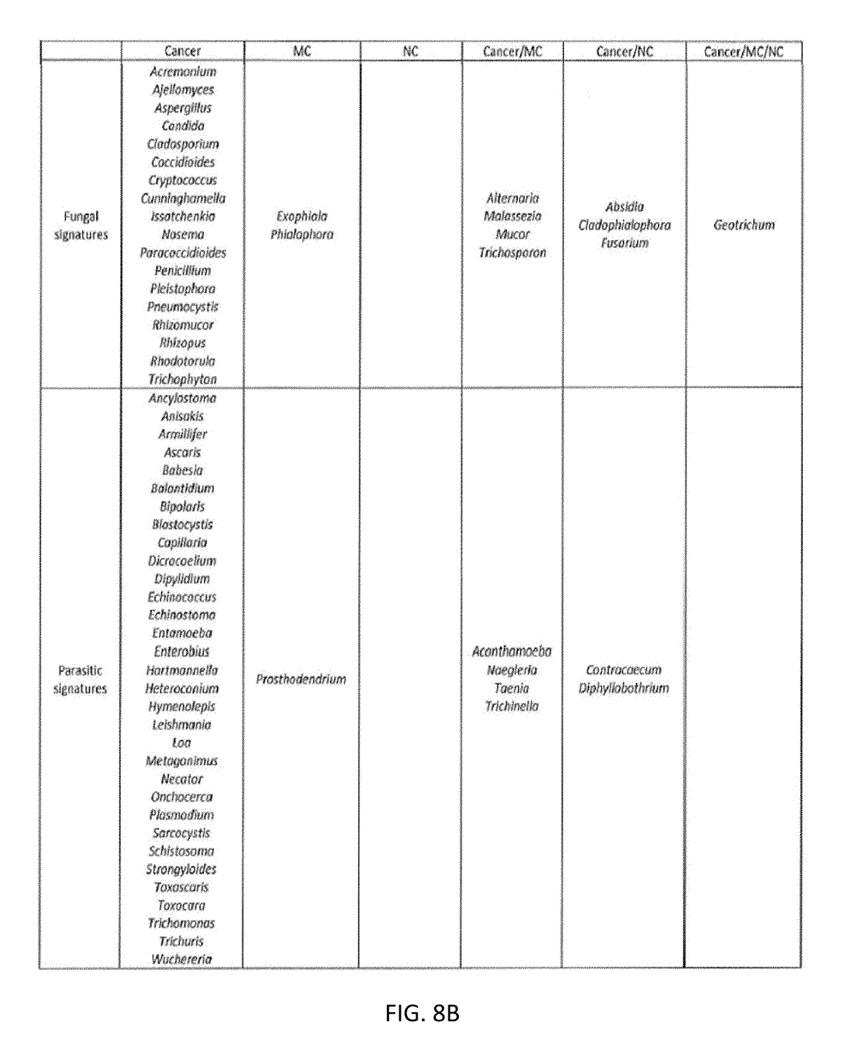

In the adjacent matched controls and in non-matched control samples, signatures of tumorigenic viral families were detected, along with other viral signatures (FIG. 1D-1E). FIG. 1G and FIGS. 8A-8B show common as well as unique viral signatures detected in ovarian cancer, when compared to the matched and non-matched controls.

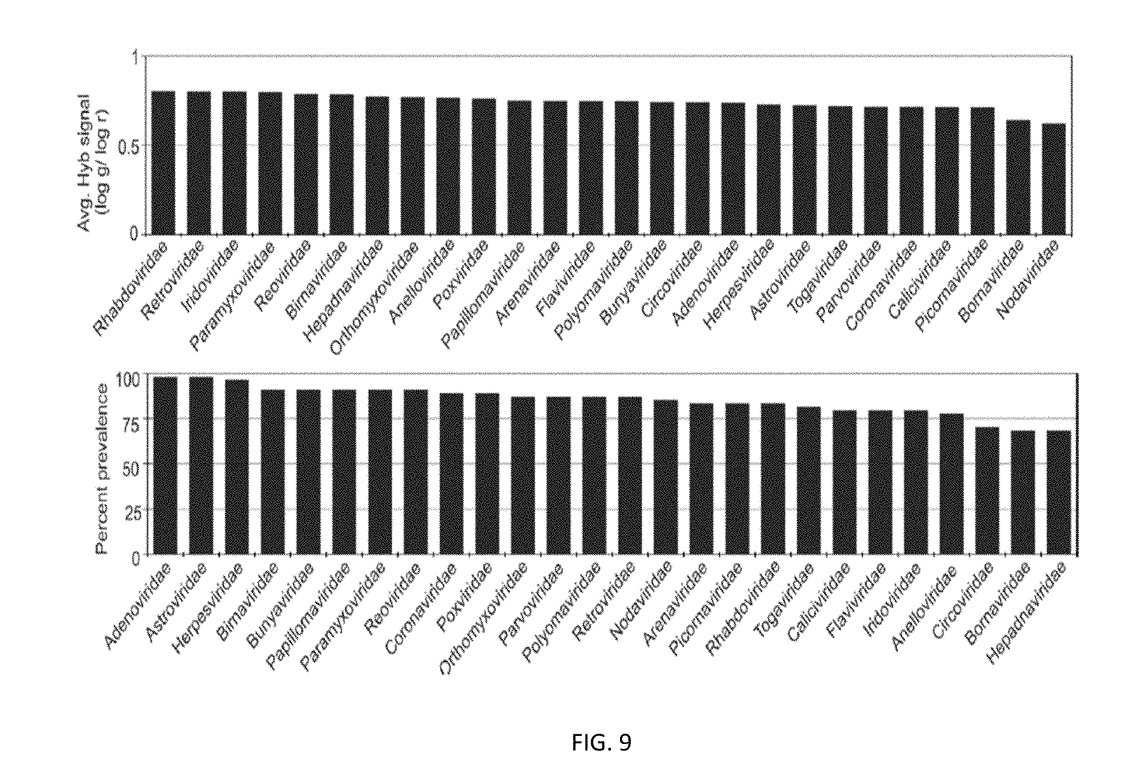

The data suggest that there is substantial perturbation of the virome which correlates with ovarian cancer. First, the average hybridization signal for the viral families detected in the cancer is lower compared to the control samples (compare FIG. 9 with FIGS. 1C-1E). Second, despite lower hybridization signal for many viruses in the cancer samples, the viral families present are quite different from controls; for example, signatures of Anelloviridae, Astroviridae, Birnaviridae, Bornaviridae, Caliciviridae, Hepadnaviridae, Iridoviridae, Paramyxoviridae, Rhabdoviridae and Togaviridae were detected at significant levels only in the cancer samples (FIGS. 8A-8B, FIG. 9) Third, among the viral families detected in both cancer and control samples, specific members of a virus family differed between cancer and controls. For example, specific molecular signatures of the high risk HPV16 and 18 were detected only in the cancer samples and not in the matched or non-matched control group. Instead the non-matched control samples showed significant detection of molecular signatures of L1 major capsid gene of HPV 41, 88, 53, and E1 gene of HPV 103 (FIG. 1F). A similar situation was detected with the poxviridae. While signatures of poxviridae that are conserved across the family were significantly detected in cancer as well as the controls (both matched and non-matched) (FIG. 1F), highly specific signatures of certain poxviruses [Monkeypox virus, Myxoma virus, Yaba monkey tumor virus (YMTV), Yaba-like disease virus (YLDV)] and parapoxviruses [(Pseudocowpox virus (PCP), Orf virus (Orf), Bovine papular stomatitis virus (BPSV)] were detected only in the ovarian cancer samples (FIG. 1F). The specific parapoxvirus signatures detected were that of IL-10 encoded by Orf virus and Bovine papular stomatitis virus, and the A-type inclusion protein of Pseudocowpox virus and Orf virus, as well as the glycoprotein of Orf virus. Specific signatures of poxviruses detected were sequences of thymidine kinase (66R) and ankyrin repeat (147R) of the tumorigenic Yaba monkey tumor virus, 3-beta-hydroxysteroid dehydrogenase of Yaba-like disease virus. Also, the majority of the Polyomavirus probes significantly detected in the ovarian cancers were that of Merkel cell Polyomaviruses which were undetectable in the controls, whereas the majority of the Polyomavirus probes detected in the controls were that of SV40, traces of which were also detected in the cancers (FIG. 1F. Among the retroviral probes detected in the majority of cancer were specific probes of Mammary Tumor Virus (MMTV) and Foamy Virus (SFV), whereas, the majority of Retroviral probes detected in the controls were of specific probes for the lentivirus subgroup of retroviruses (FIG. 1E). Interestingly, the detection of Herpesviridae probes identified HHV2 with high significance in the non-matched control compared to the cancers. However, the cancer samples showed detection for conserved and specific probes of HHV6A and IHHV6B which were undetectable in the controls. Other herpesviridae probes of HHV4, HHV5 and HHV8 were detected in both cancer and non-matched control samples (FIG. 1F).

The data as a whole suggest that specific viral signatures were dramatically altered in the cancer tissue. Some signatures appeared only in the cancer or have significantly increased hybridization intensity, while others are decreased compared to the surrounding tissue. Several points must be kept in mind when considering these data: 1) the tumor microenvironment may provide advantages for the persistence of some viruses, thus promoting their presence in the cancer. Hence, their presence need not be related to the cause of the cancer. Similarly, the appearance of a virus in the matched control and not the cancer may suggest that the tumor microenvironment is inhibitory for persistence of the virus. 2) The probes may also be detecting relatives or variants of known viruses from which the probes were derived. For example, specific probes for lentiviruses including HIV-1 were positive in the analysis of control samples. These were de-identified samples, however it is doubtful that these patients were HIV positive but suspected that the probes were likely detecting the presence of a related, uncharacterized human lentivirus.

### Example 3: Identification of Bacterial Signatures Associated with Ovarian Cancer

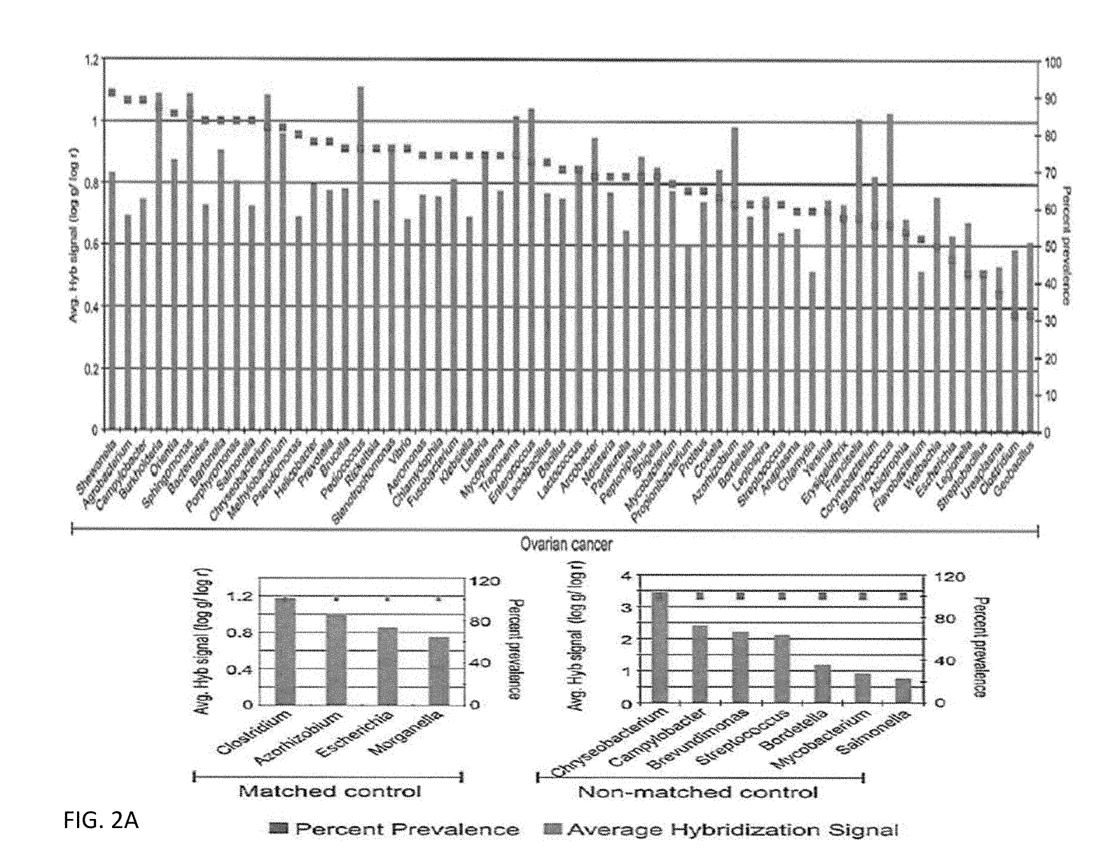

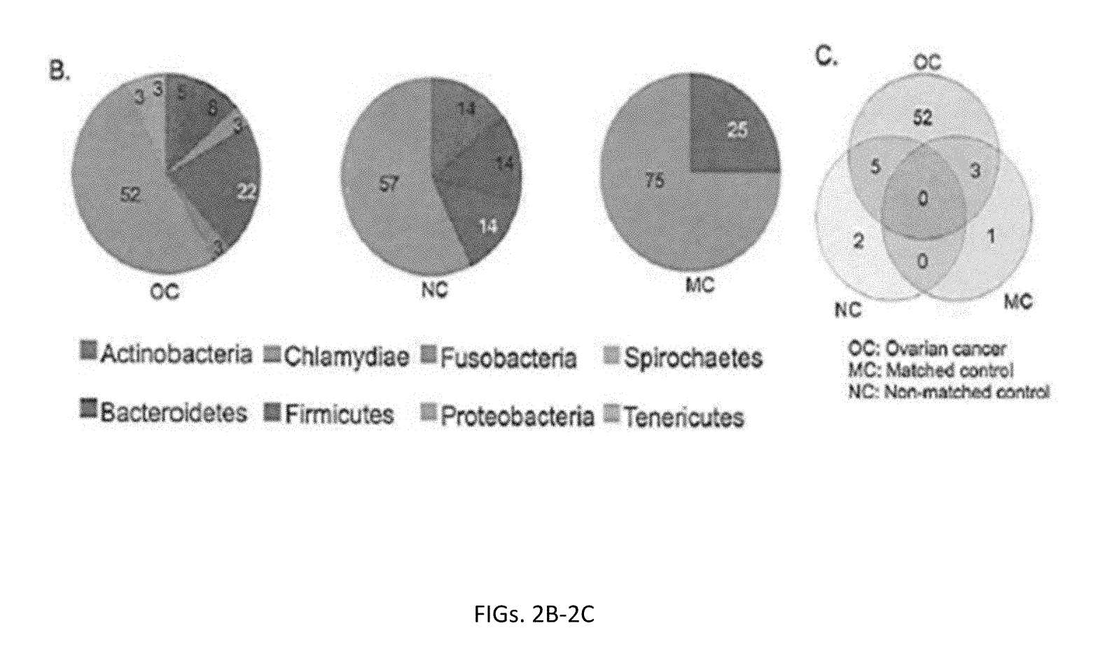

Similar to that seen with the viruses, the bacterial signatures were dramatically altered from those of matched controls and non-matched controls. The specific bacterial signatures detected in the cancer and the matched and non-matched samples are shown in FIG. 2A according to their decreasing prevalence. Two predominant bacterial phyla were detected in the ovarian cancer samples screened. They were Proteobacteria (52%), followed by Firmicutes (22%) (FIG. 2B). Other phyla were also detected at lower percentages including Bacteroidetes, Actinobacteria, Chlamydiae, Fusobacteria, Spirochaetes and Tenericutes in the cancer samples. Signatures of Proteobacteria and Firmicutes were also detected significantly in the matched control samples screened, and that of Proteobacteria, Actinobacteria, Bacteroidetes and Firmicutes were detected significantly in the non-matched control samples (FIG. 2B). Many more bacterial signatures were significantly detected in the cancer samples compared to the controls. The signatures associated only with the ovarian cancer samples are listed in FIGS. 2A-2B and FIGS. 8A-8B. The different bacterial signatures, unique or common to the control and ovarian cancer samples are listed in FIGS. 8A-8B and represented in FIG. 2C.

While signatures of Pediococcus were detected with the highest hybridization signal in the ovarian cancer samples screened, followed closely by that of Burkholderia, Sphingomonas, Chryseobacterium, Enterococcus, Staphylococcus, Treponema and Francisella [(log g/log r)>1], Shewanella signatures were detected with the highest prevalence in 91% of the cancers (FIG. 2A). The majority of the bacterial signatures detected in the cancers had high prevalence, except for signatures of Escherichia, Legionella, Streptobacillus, Ureaplasma, Clostridium, Geobacillus which were detected in less than 50 percent of the cancer samples screened (FIG. 2A). There were no common bacteria between all 3 types of samples (FIG. 2C, FIGS. 8A-8B). However, 5 agents were shared between the cancer and non-matched controls, and 3 agents between the cancer and matched controls (FIG. 2C, FIGS. 8A-8B). 52 unique bacterial agents were detected predominantly in only the cancer (FIG. 2C, FIGS. 8A-8B).

### Example 4: Identification of Fungal Signatures Associated with Ovarian Cancer

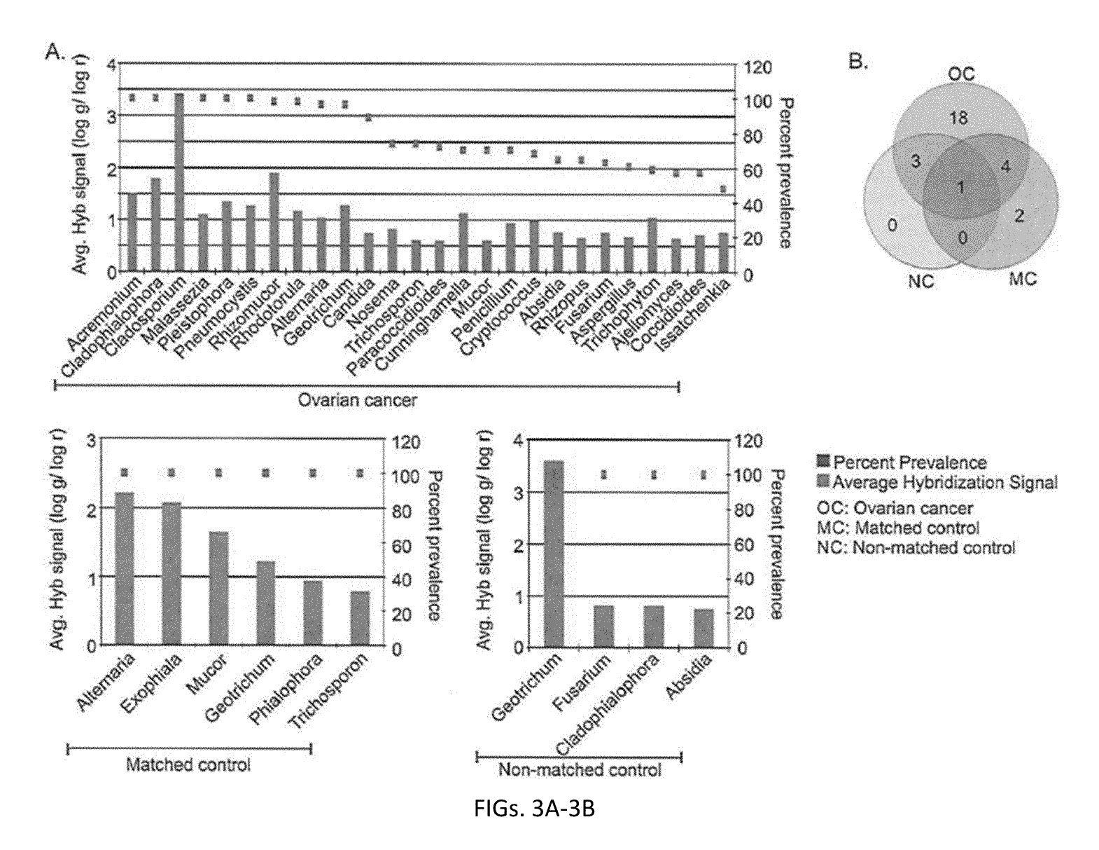

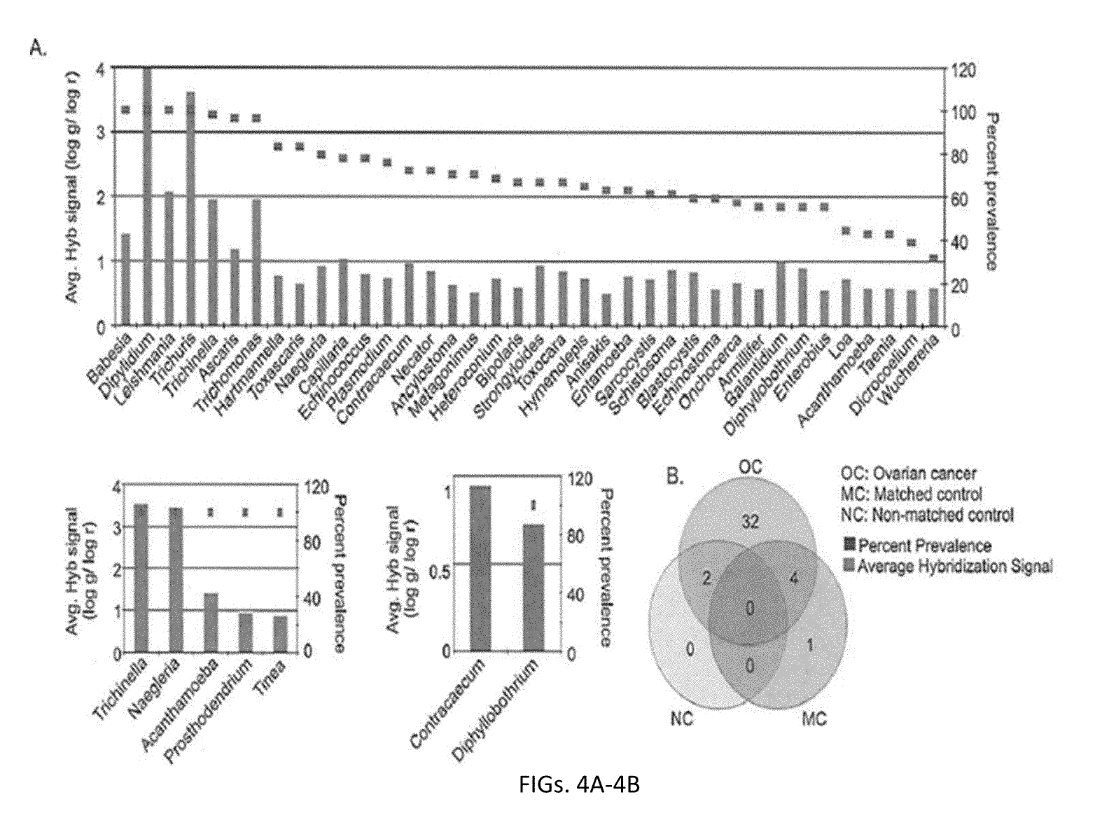

The pathogen screen for fungal signatures again suggests a significant perturbation of the microbiome in the tumor. The fungal signatures detected in the ovarian cancer and controls are shown according to their decreasing prevalence in FIG. 3A. Fungal signatures that were detected only in the ovarian cancer samples and interestingly not found associated with the controls are listed (FIGS. 8A-8B, FIGS. 3A-3B). 18S rRNA signatures of Cladosporium were detected in all the ovarian cancer samples with the highest hybridization signal. Signatures of Pneumocystis, Acremonium Cladophialophora, Malassezia and microsporidia Pleistophora were also detected significantly in all the ovarian cancer samples screened (FIG. 3A). Signatures of Rhizomucor, Rhodotorula, Alternaria, Geotrichum were also found to be associated with more than 95% of the ovarian cancer samples screened (FIG. 3A). It should be noted that the signature of Geotrichum was also detected in all the control samples (FIGS. 8A-8B, FIG. 3A). Therefore the associated fungal agents appeared to be dominant in the ovarian cancer with only Geotrichum common among the cancer and controls. This suggested that they may be more tightly associated in this particular microenvironment than previously predicted.

### Example 5: Identification of Parasitic Signatures Associated with Ovarian Cancer

The parasitic signatures detected in the ovarian cancer and controls are shown in FIG. 4A, according to their decreasing prevalence. The parasitic signatures significantly detected in the cancer samples versus the non-matched controls were much higher compared to parasitic signatures detected significantly in the controls versus cancer, once again suggesting a marked perturbation of the tumor microbiome. The parasitic signatures detected only in the ovarian cancer samples are listed in FIG. 4A and FIGS. 8A-8B. All of the tumor samples showed a high hybridization signal (log g/log r>2) for the 28S rRNA signature of Dipylidium. A high hybridization signal for the 18S rRNA signatures of Trichuris and Leishmania was also found in all of the ovarian cancer samples (FIG. 4A). The 18S rRNA signatures of Babesia were also significantly detected in all the ovarian cancer samples, although with a relatively moderate hybridization signal (log g/log r>1, <2) (FIG. 4A). 18S rRNA signatures of Trichinella, Ascaris, and Trichomonas were detected in >95% of the ovarian cancer samples screened, also with a moderate hybridization signal intensity (log g/log r>2) (FIG. 4A). The other parasitic signatures detected in the ovarian cancer listed in FIG. 4A were detected with lower hybridization signal intensity (log g/log r<1), although with high prevalence except for signatures of Loa loa, Acanthamoeba, Taenia, Dicrocoelium, Wuchereria which were detected in less than 45% of the ovarian cancer samples screened. Signatures of 4 parasites that were detected in the cancer samples were also found in the adjacent matched control samples, these include Acanthamoeba, Naegleria, Taenia and Trichinella (FIG. 4A, FIGS. 8A-8B). However, they were not detected in the non-matched controls (FIG. 4A).

### Example 6: Hierarchical Clustering of the Ovarian Cancer Samples

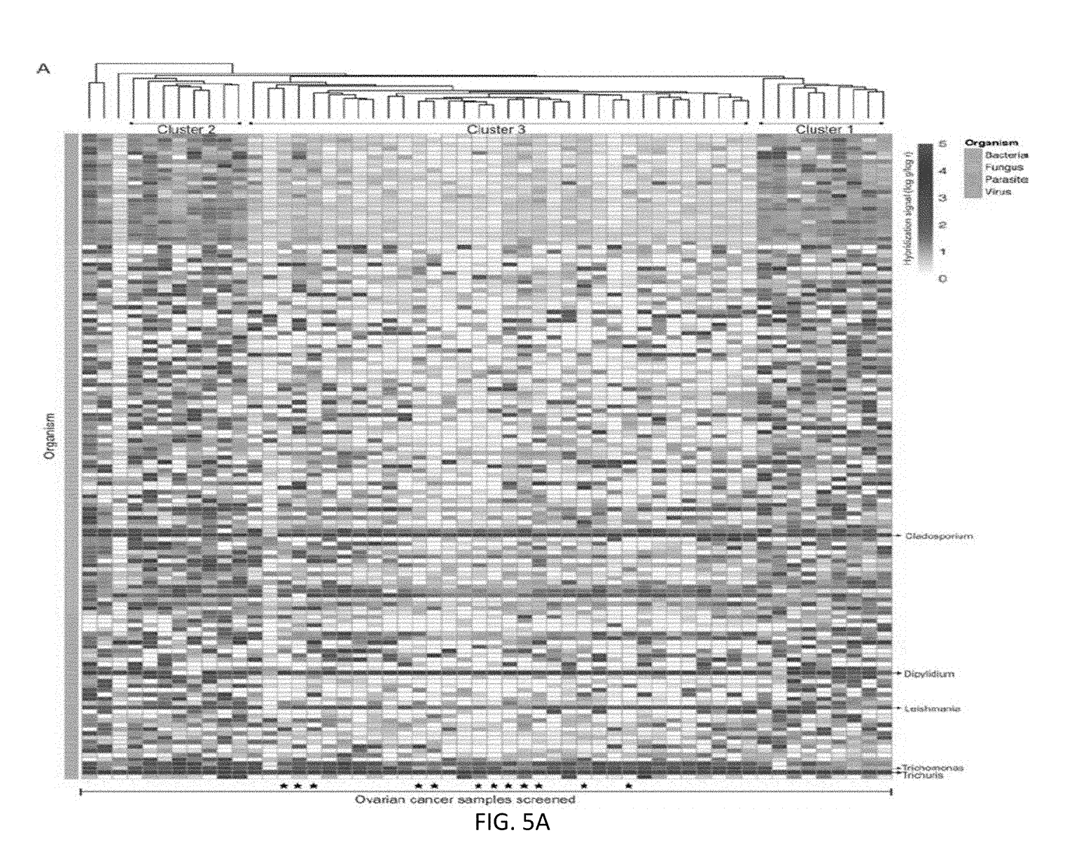

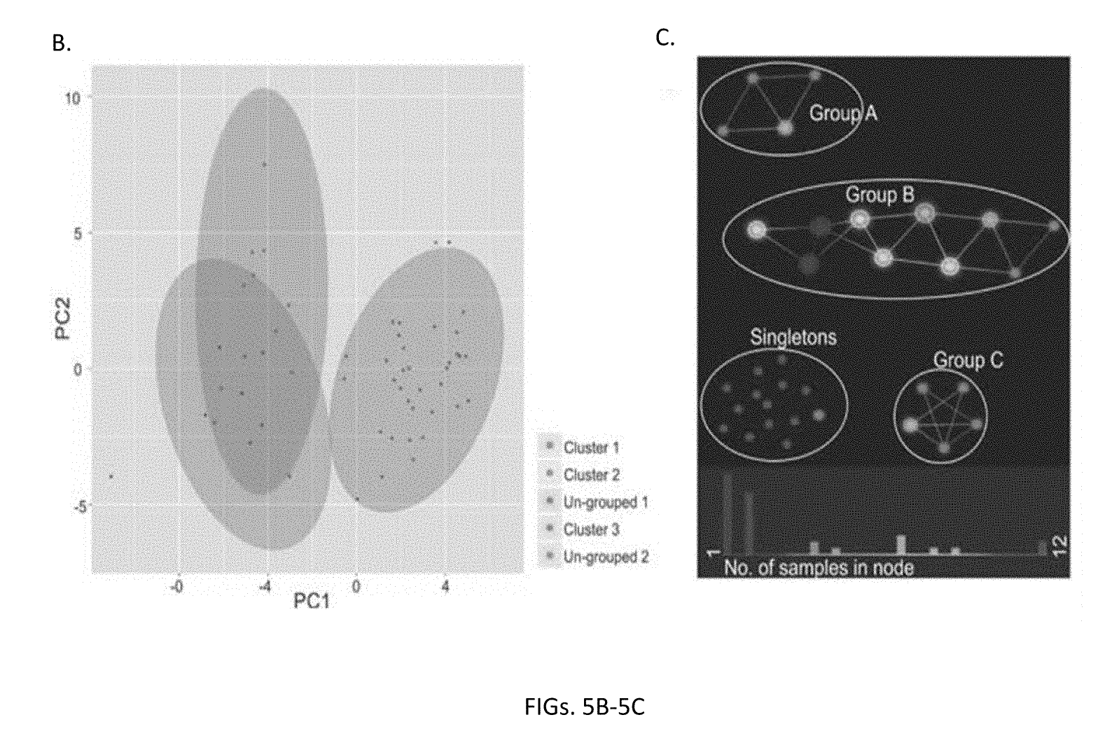

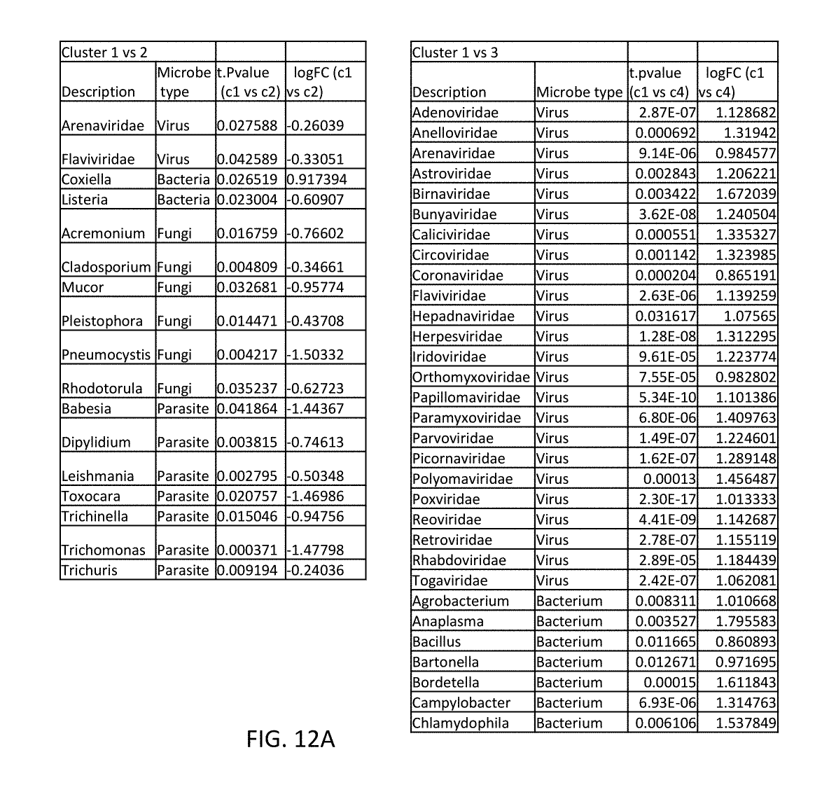

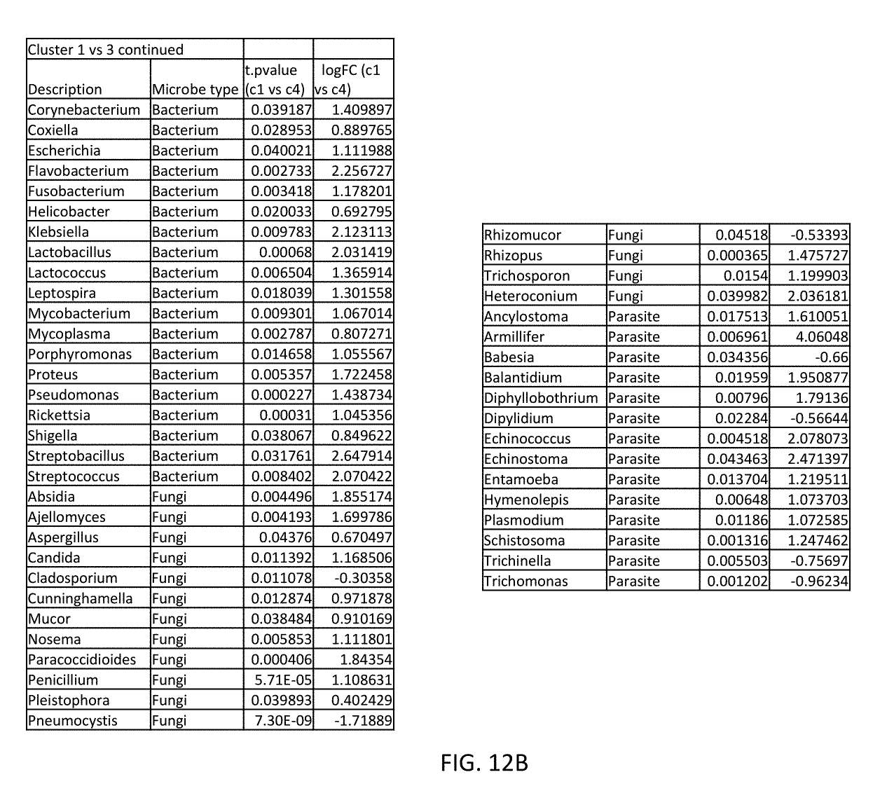

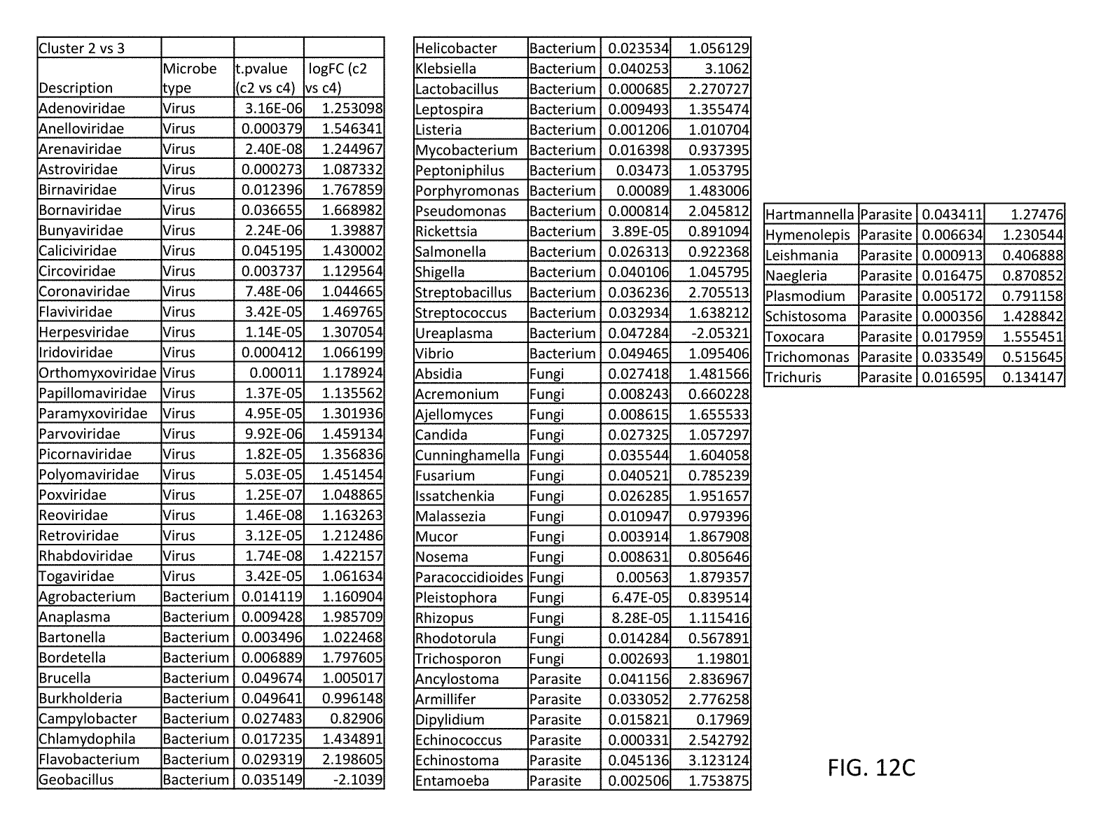

Hierarchical clustering analysis of the ovarian cancer samples compared the similarity of the overall microbiome signatures detected in each ovarian cancer sample and clustered the samples together based on common microbiome similarity (FIGS. 5A-5B). While some samples did not group into a cluster (namely un-grouped 1 and 2), the majority of the samples grouped into three distinct clusters, namely cluster 1, 2 and 3, with cluster 3 samples showing significant differences in detection of several viral and microbial signatures compared to the samples of cluster 1 and 2. FIGS. 12A-12C shows the significant differences in microbial detection between the clusters. Ovarian cancer samples of cluster 1 and 2 showed significant differences in the detection of 2 viral agents (Arenaviridae and Flaviviridae) and bacterial agents (Coxiella and Listeria) signatures, and few fungal (Acremonium, Cladosporium, Mucor, Pleistophora, Pneumocystis and Rhodotorula) and parasitic (Babesia, Dipylidium, Leishmania, Toxocara, Trichinella, Trichomonas and Trichuris) signatures. These signatures are all of higher intensities in cluster 2 than 1. On the other hand, ovarian cancer samples of cluster 3 had significantly less detection of almost all the viral and several microbial signatures mentioned in FIGS. 12A-12C.

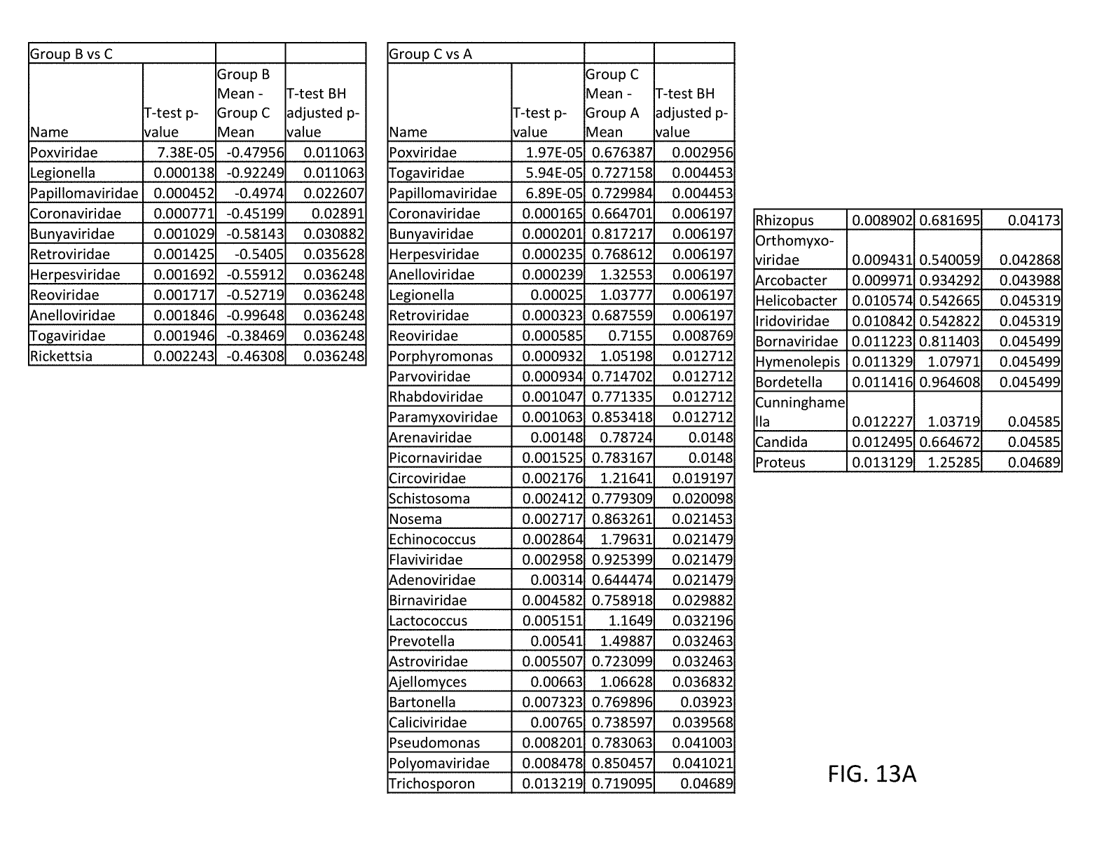

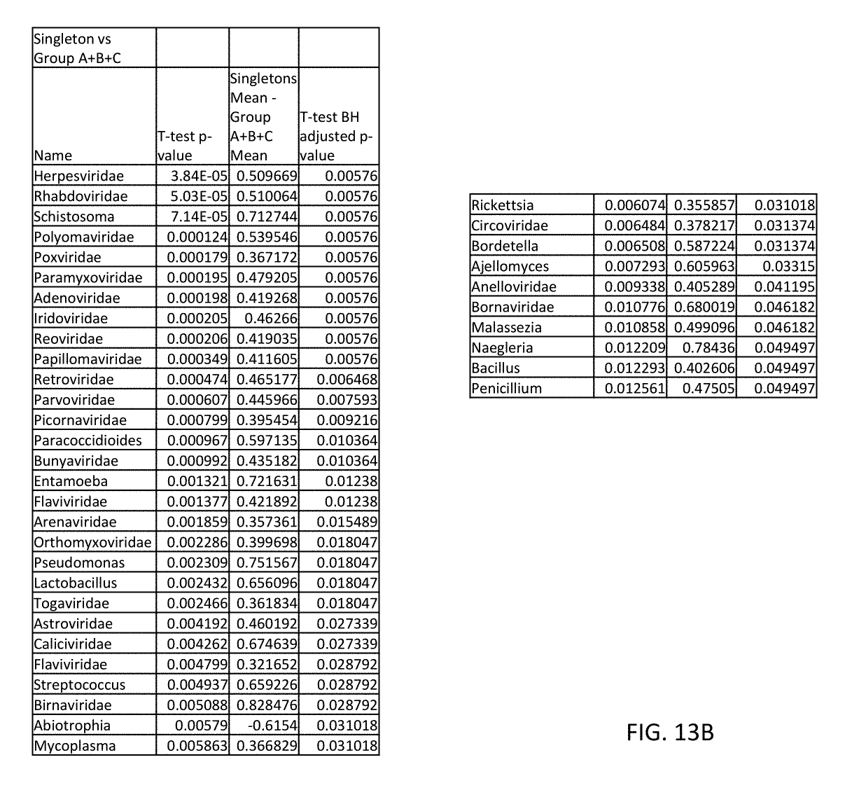

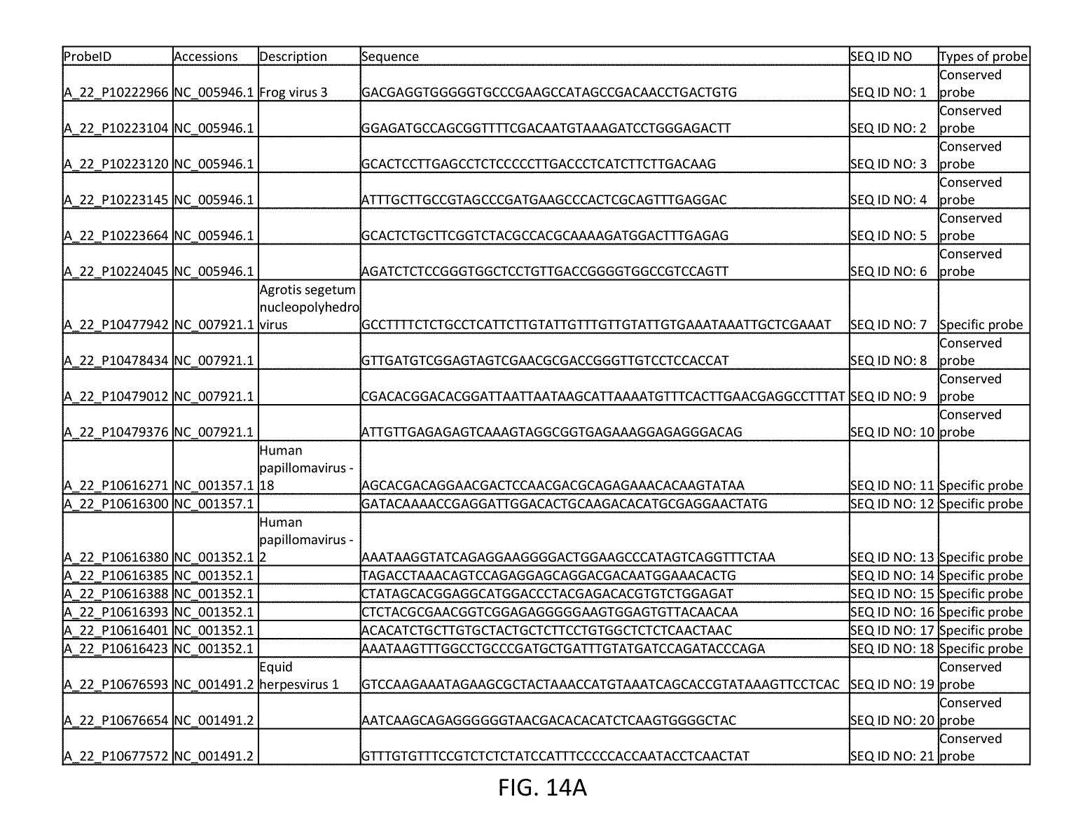

Based on the topological analysis, the ovarian cancer samples clustered into 3 groups (A, B and C), while some could not be grouped together (singletons) (FIG. 5C). FIGS. 13A-13B show significant differences in microbial detection in each group. Group B had significantly higher detection of the following signatures compared to Group A: viral signatures of Coronaviridae, Astroviridae, Togaviridae, Reoviridae, Papillomaviridae, Poxviridae, Bunyaviridae, Picornaviridae, Paramyxoviridae, Bornaviridae, Birnaviridae, Rhabdoviridae, Caliciviridae, Arenaviridae and Flaviviridae; along with certain bacterial signatures of Porphyromonas, Anaplasma, Azorhizobium, Corynebacterium, Arcobacter, Lactococcus, Methylobacterium, Shigella, Proteus, Brucella, Ureaplasma and Prevotella; fungal signatures of Absidia, Trichophyton, Ajellomyces, Geotrichum and Candida; and parasitic signatures of Ascaris, Bipolaris, Acanthamoeba, Sarcocystis, Balantidium, Echinostoma, Dicrocoelium and Wolbachia. Group C differed from group B in having significantly higher signatures of mainly viral families of Poxviridae, Papillomaviridae, Coronaviridae, Bunyaviridae, Retroviridae, Herpesviridae, Reoviridae, Anelloviridae and Togaviridae and bacterial signatures of Rickettsia and Legionella compared to Group B. Group C differed from Group A in having significantly higher detection of the viral signatures of Poxviridae, Togaviridae, Papillomaviridae, Coronaviridae, Bunyaviridae, Herpesviridae, Anelloviridae, Retroviridae, Reoviridae, Parvoviridae, Rhabdoviridae, Paramyxoviridae, Arenaviridae, Picornaviridae, Circoviridae, Flaviviridae, Adenoviridae, Birnaviridae, Caliciviridae, Polyomaviridae, Orthomyxoviridae, Iridoviridae, Bornaviridae, Astroviridae; bacterial signatures of Legionella, Porphyromonas, Lactococcus, Prevotella, Bartonella, Pseudomonas, Arcobacter, Helicobacter, Bordetella and Proteus; fungal signature of Nosema, Ajellomyces, Rhizopus, Cunninghamella, Candida, Trichosporon and parasitic signature of Schistosoma, Echinococcus and Hymenolepis. The cancer samples which could not be grouped into a cluster (Singletons) showed significant differences in the detection of certain viral and microbial signatures than the rest of the clustered samples (FIGS. 13A-13B). The bacterial signature of Abiotrophia was detected significantly higher in the grouped ovarian cancer samples than the ungrouped singletons. However, in the singletons compared to the grouped samples (Group A+B+C) there was significantly higher detection of most viral signatures (except for Hepadnaviridae and Nodaviridae), bacterial signatures of Pseudomonas, Lactobacillus, Streptococcus, Abiotrophia, Mycoplasma, Rickettsia, Bordetella and Bacillus; fungal signatures of Paracoccidioides, Ajellomyces, Malassezia and Penicillium; and parasitic signatures of Schistosoma, Entamoeba and Naegleria.

### Example 7: PathoChip Screen Validation and Detection of Viral Insertions in Human Chromosomes of Ovarian Cancer Cells

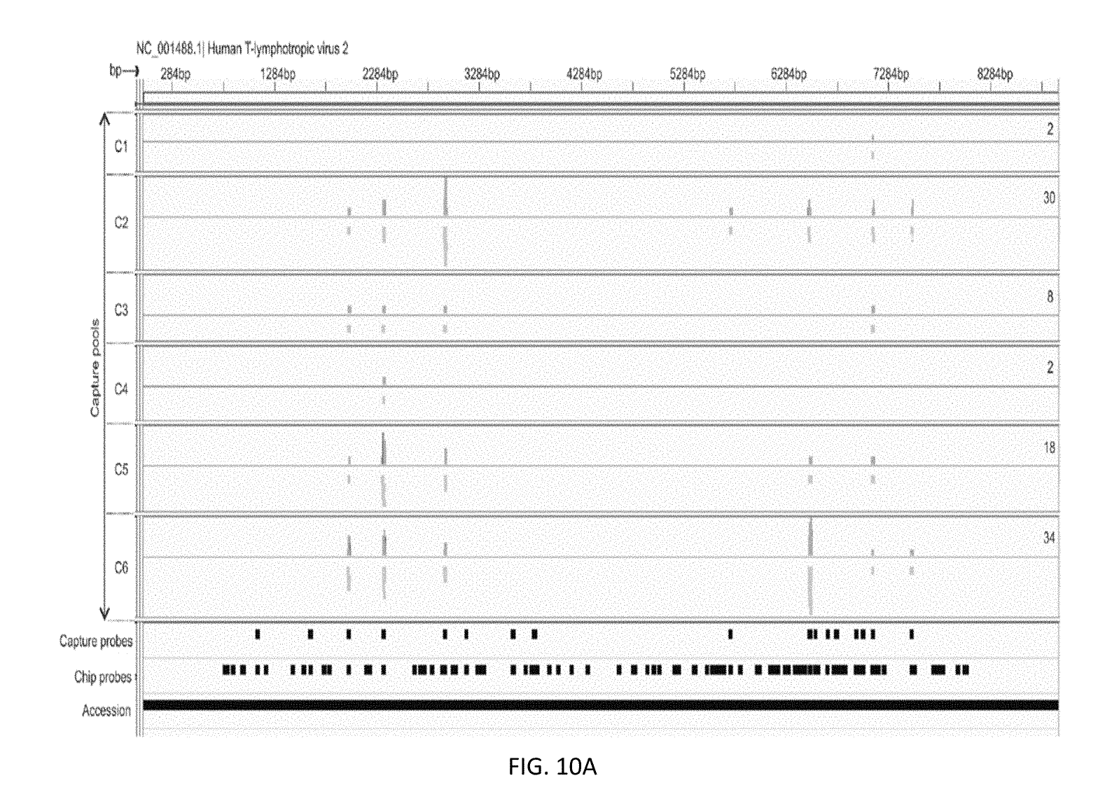

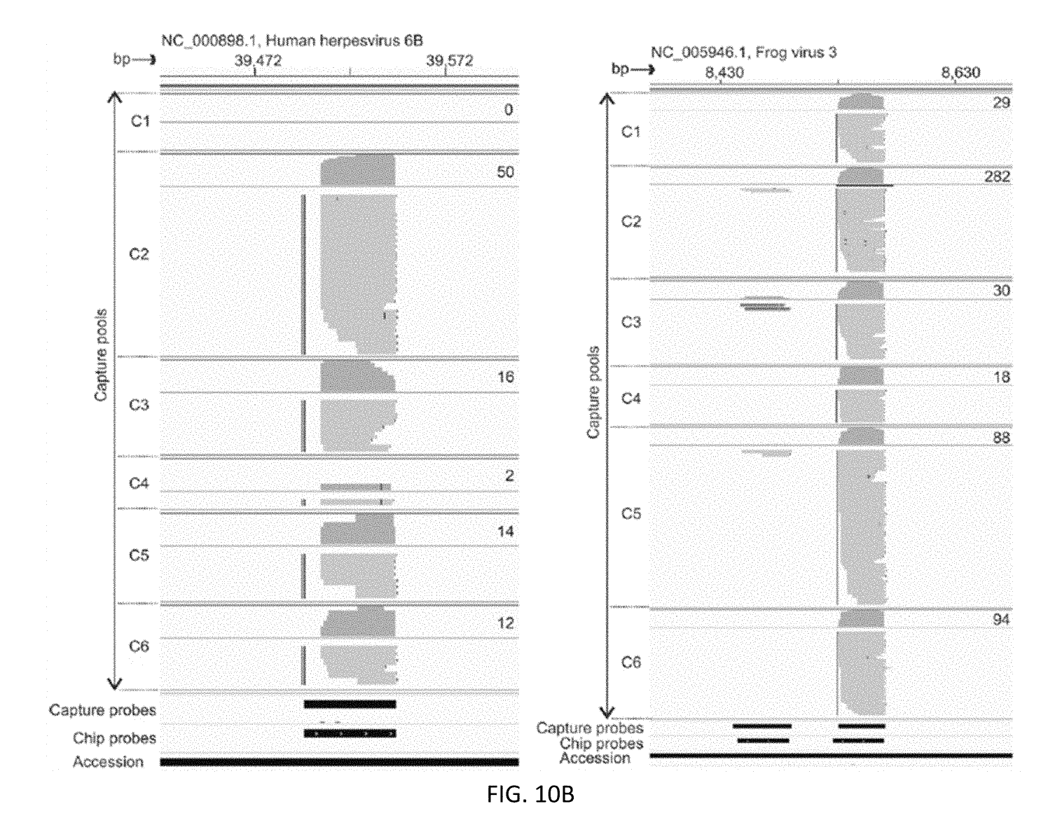

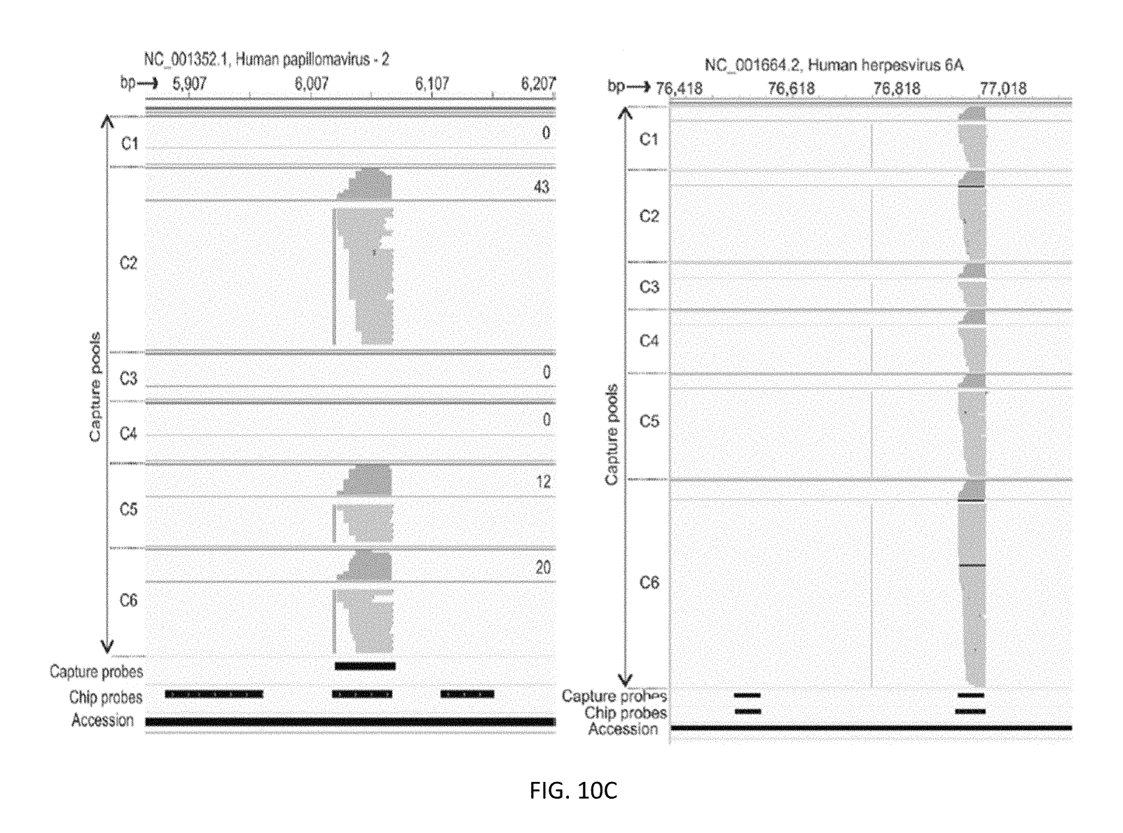

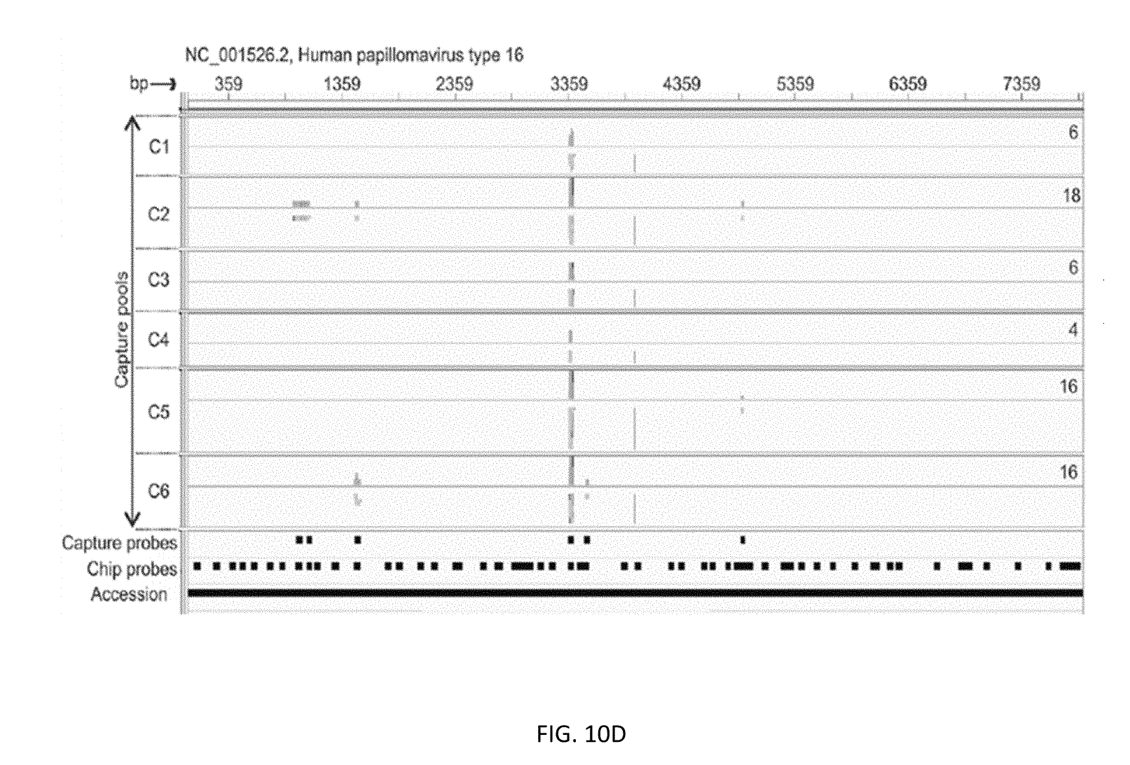

Probes of certain viruses, which were detected positive in the PathoChip screen were used as a target reagent (FIGS. 14A-14F, SEQ ID NOs. 1-94) to capture the genomic sequences of amplified products of the pooled ovarian samples. The selected targets were then subjected to next generation sequencing. The sequences, when aligned to the PathoChip metagenome, showed that they aligned at or near the capture probe locations, thus validating the PathoChip screen results (FIGS. 6A-6B, FIGS. 10A-10D). The sequence alignments to the PathoChip metagenome were visualized using the Integrative Genomics Viewer (IGV) program. Capture probes of Yaba Monkey Tumor virus, HTLV-2, HHV6a, Human adenovirus D, HPV16, HPV18, HPV2 and Iridovirus (Frog virus 3) also hybridized to and captured the viral sequences from the ovarian cancer samples (FIGS. 10A-10D). The YMTV sequence identified the g52R ORF.

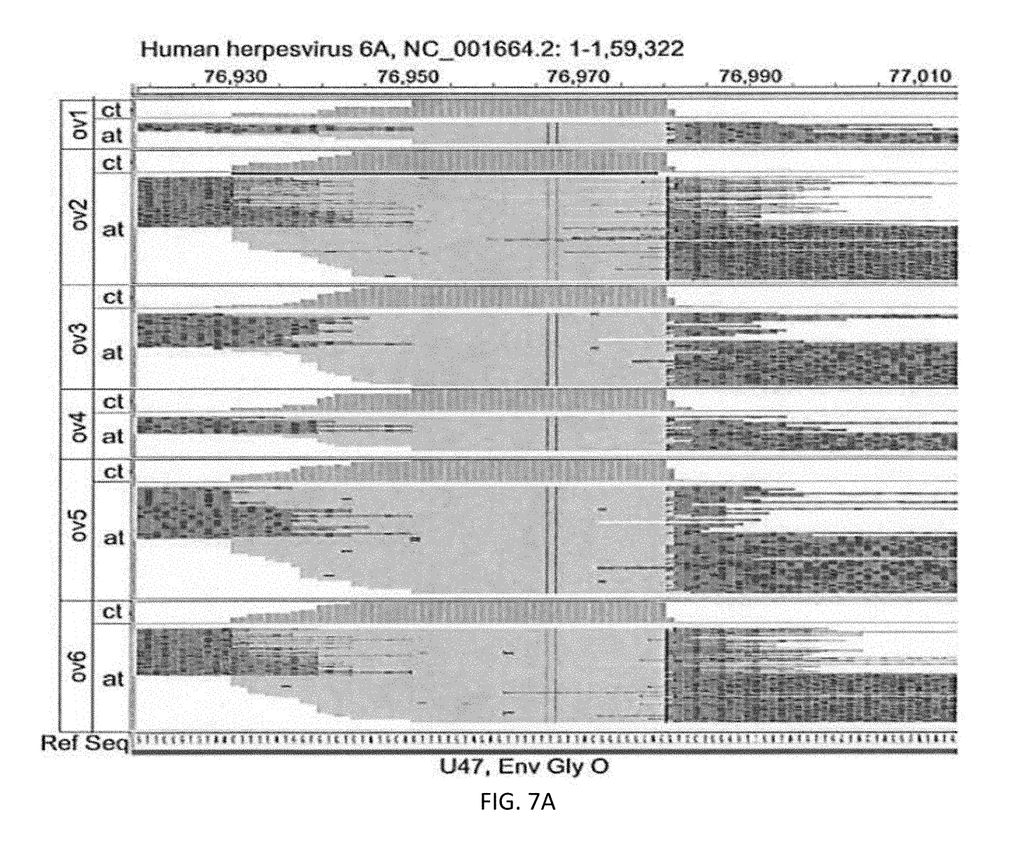

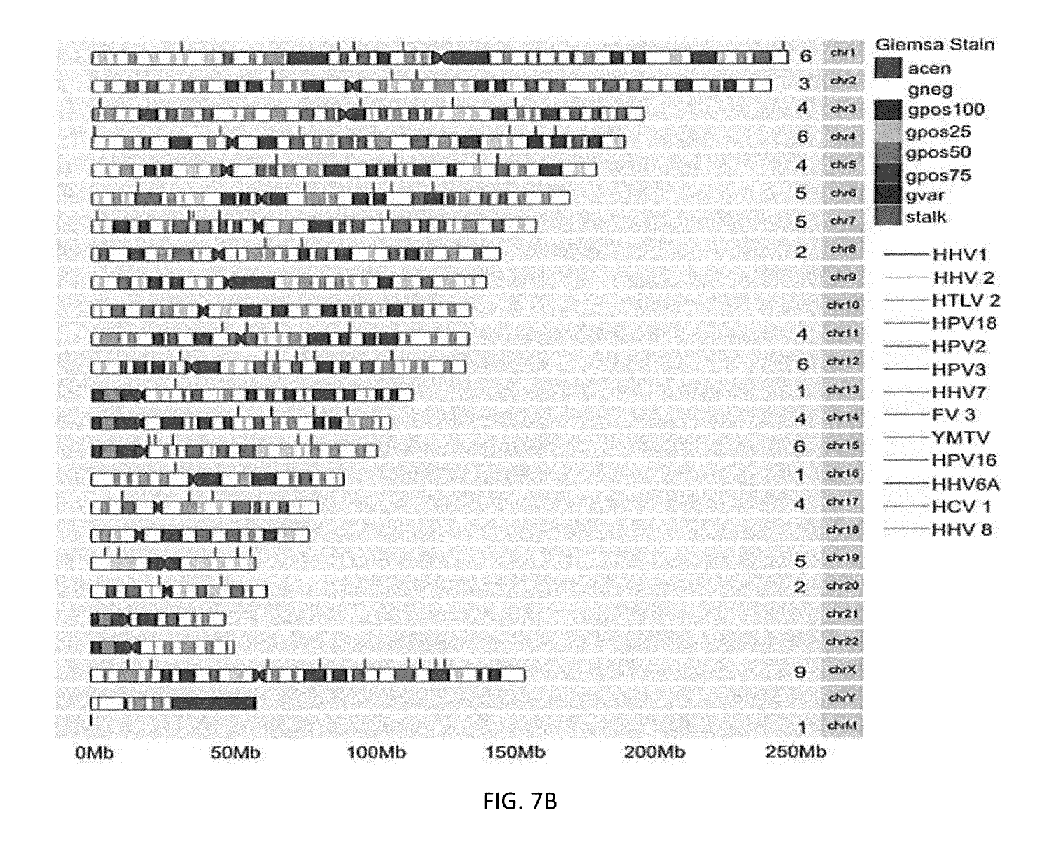

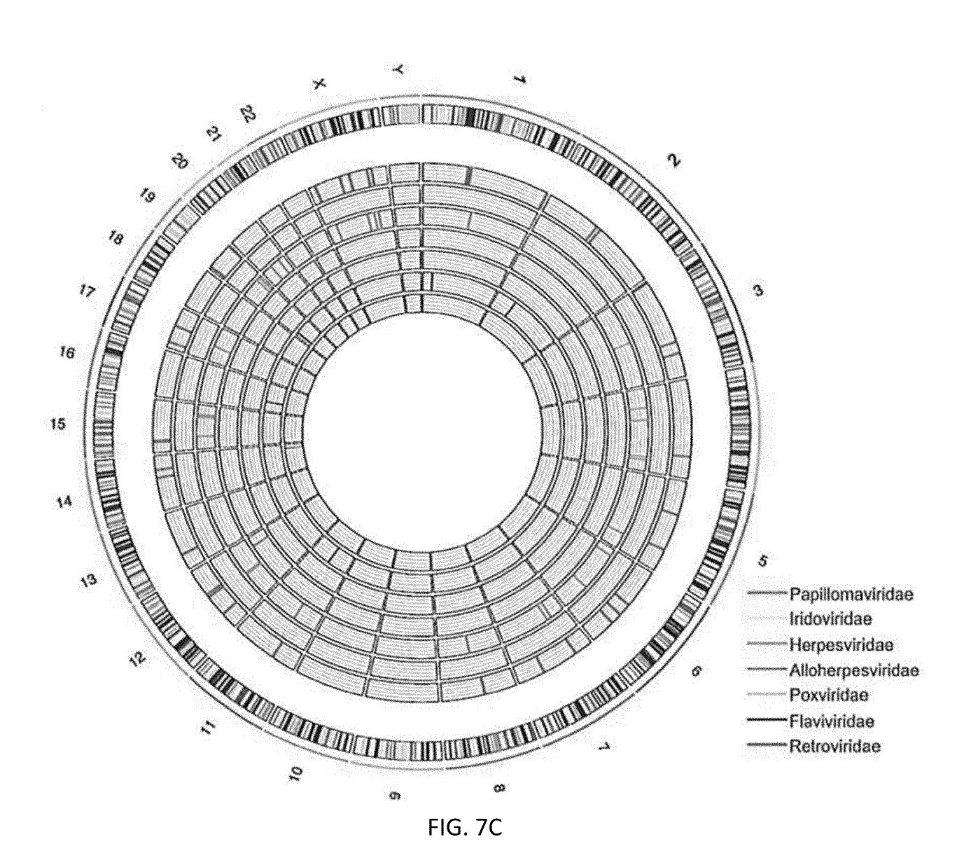

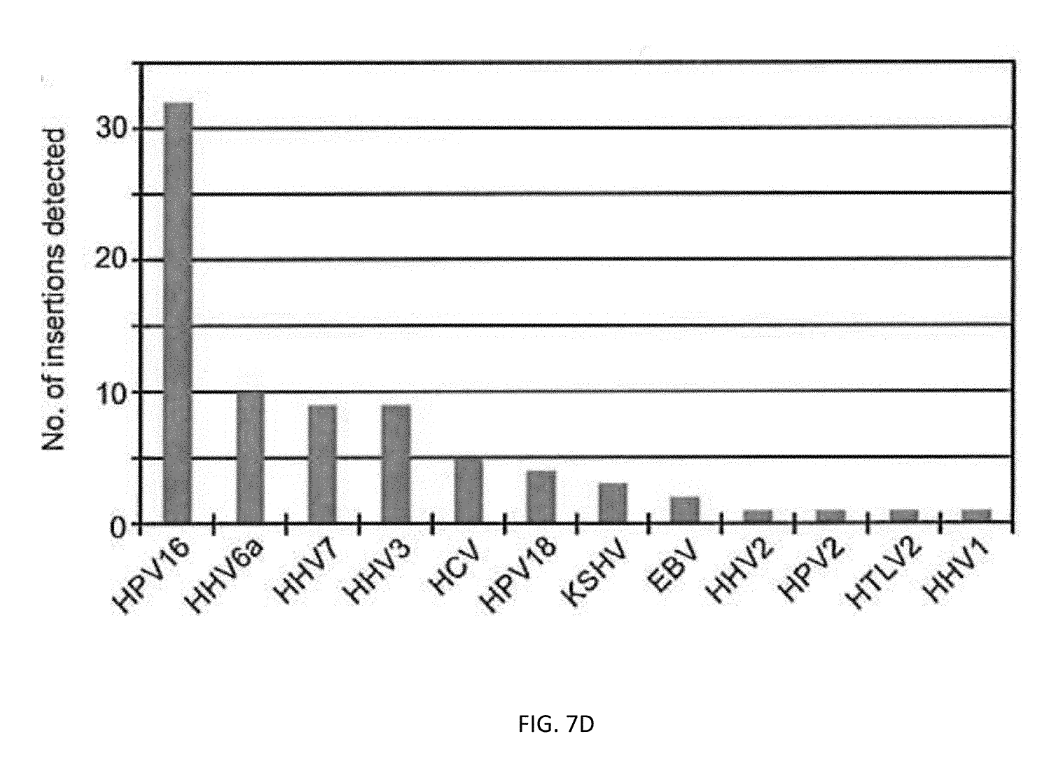

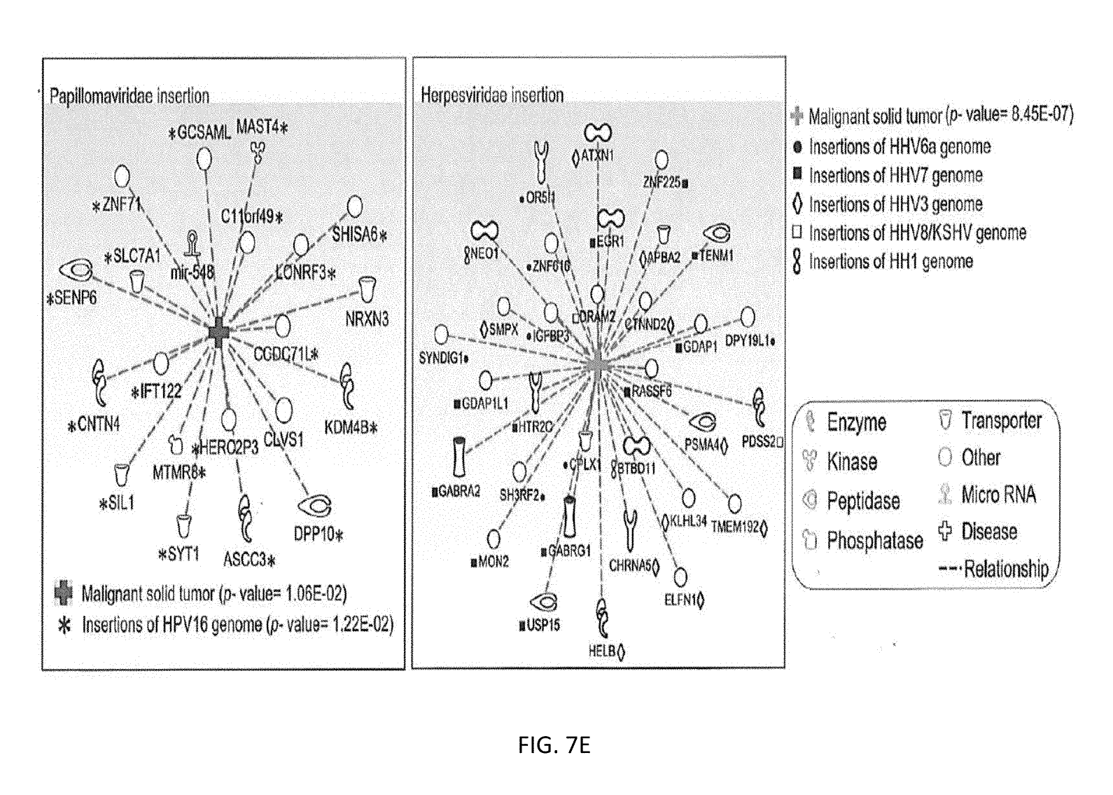

It was determined from the analyses that there were certain viral genomic integrations in the host chromosomes (FIGS. 7A-7E). Regions of some of the sequences that aligned to the PathoChip metagenome were identified to contain soft-clipped segments, which could not be aligned to the metagenome (FIG. 7A). However, these sequence segments did map to the human genome indicating specific sites of microbial genomic integrations in the human genome. The highest number of viral integration sites were detected in the somatic human chromosomes for HPV16 with over 30 integrations (FIGS. 7B-7D) with 5 integrations in the X-chromosome and 3 in chromosome 6. This was followed by HHV6a, HHV7 and HHV3 with less than 10 integrations (FIGS. 7B-7D). The genes at or proximal to the detected viral integrations were then subjected to Ingenuity Pathway Analysis (IPA) software to determine if those genes were associated with the development or association with cancer (FIG. 7E). The software calculates the significance of such associations.

### Example 8: Identification of HPV Insertions in Ovarian Cancer

Examination of the HPV insertion data showed integration of HPV16 genomic sequences around the polyA sequence of E5 (co-ordinate 4184-4213 of NC_001526.2), which was known to be hotspot for integration, integrated at intronic and intergenic regions of a number of human somatic chromosomes. HPV16 integration was seen at the intronic regions of MAST4 (chr5), IFT122 (chr3), CYFIP1 (chr15), EEPD1 (chr7), C11orf49 (chr11), SYT1 (chr12), HERC2P3 (chr15), ZNF71 (chr19), ASCC3 (chr6), GCSAML (chrl), MTMR8 (chrX), SIL1 (chr5), CNTN4 (chr3), KDM4B (chr19), METTL20 (chr12), DPP10 (chr2) and SENP6 (chr6). HPV16 genomic integrations were also detected at about 29 Kb upstream of the SLC7A1 gene (chr13), 15 Kb upstream of the SHISA6 (chr17), 56 Kb upstream of the ncRNA gene LOC101928137 (chr12), 21 Kb upstream of GS1-600G8.3 (chrX), 33 Kb upstream of CCDC71L (chr7), 12 Kb upstream of LONRF3 and 81 Kb downstream of ncRNA LINC01285 (chrx), 26 Kb downstream of LOC644172, and 53 Kb upstream of LRRC37A4P (chr17).

Regions from the coding sequence of the E1 gene of HPV18 were found to be integrated at the intronic regions of ncRNAs LOC100131564 (chr1) and MIR548AZ (chr14), as well as at intergenic regions of the mitochondria chromosome. Genomic regions of the L1 gene of HPV18 were also detected at the intronic region of the NRXN3 gene (chr14). Among other HPV insertions, the coding sequence of the L1 gene of HPV2 was detected at the intronic region of the CLVS1 gene in chr8. Of the 36 genes that could be affected due to HPV genomic insertions, 21 were found be significantly associated with malignant solid tumors (p value=1.06E-02) as predicted by Ingenuity Pathway Analysis software (FIG. 7E). Of the probable 32 genes that could be affected by HPV 16 genomic insertion at or near those genes, 18 of them, namely ASCC3, C11orf49, CCDC71L, CNTN4, DPP10, GCSAML, HERC2P3, IFT122, KDM4B, LONRF3, MAST4, MTMR8, SENP6, SHISA6, SILL SLC7A1, SYT1 and ZNF71 genes were found to be significantly associated with malignant solid tumors (p value=1.22E-02) (FIG. 7E). Among the other HPV genomic insertions detected that could affect gene expression of 4 others, 2 genes, MIR548AZ and NRXN3 were affected by HPV18 genomic integration at the intronic region and the CLVS1 gene which was affected by intronic integration of HPV 2 were also found to be significantly associated with malignant solid tumor formation (FIG. 7E).

### Example 9: Herpesvirus Insertions within the Ovarian Cancer Chromosomes

Among the herpesviridae genomic insertions detected were that of HHV6a, KSHV, Herpesvirus 4, Herpesvirus 1, Herpesvirus 2, HHV3 and HHV7 (FIGS. 7B-7D). Of the 36 genes, at or proximal, many herpesviral genomic integrations were detected. 32 were significantly associated with tumorigenesis (p-value=8.45E-07) as predicted by IPA software (FIG. 7E). Coding sequence (CDS) of the U47 gene of HHV6a (NC_001664 at 76981) which encodes for the envelope glycoprotein O, involved in virion morphogenesis was found to be integrated at various regions of the host chromosome (chr), namely at the intronic region of SH3RF2 gene (chr 5), ZNF616 gene (chr19), SYNDIG1 gene (chr20), CPLX1 (chr4), at the exonic region of OR5I1 (chrl 1), at the downstream of DPY19L1 (chr7), and at certain intergenic regions like 58 Kb upstream of LHX1 and 25 Kb upstream of IGFBP3 (chr7). Most of these genes which may be affected due to HHV6a genomic insertions at or near the genes except for LHX1 were found to be significantly associated with different cancers (p-value=8.54E-04) (FIG. 7E).

Many of the capture probes used were from the conserved sequences of Herpesviruses (FIGS. 14A-14F), and these conserved probes allowed for detection of Herpesvirus 4, Herpesvirus 1, Herpesvirus 2 genomic sequences integrated at various somatic chromosomal locations; CDS of ORF71 of Herpesvirus 4 was detected integrated within the intergenic region of chromosome M, genomic sequence matching to the CDS of ORF18 of Herpesvirus 1 was found integrated at the intronic region of BTBD11 (chr12), and genomic sequence of the CDS of UL42 gene which encodes the DNA polymerase processivity subunit for DNA replication was found to be integrated at the intronic region of the NE01 gene (chr15). Both of these genes were found to be associated with endometrioid carcinoma (p-value=2.27E-02) (FIG. 7E).

CDS of vlRF-2 (viral interferon regulatory factor 2) of HHV8 was found to be integrated 57 Kb downstream of DRAM2 (chr 1), while tegument protein coding sequence was seen to be integrated at the intronic region of the PDSS2 tumor suppressor gene (chr6). Again, both of these genes were associated with cancer (FIG. 7E).

Interestingly, CDS of ORF6 that encodes the helicase-primase subunit for DNA replication of the HHV3 sequence integrated at multiple sites of different chromosomes. This region could be a hotspot for HHV3 integrations within the host chromosomes. Insertions were detected at the intronic regions of TMEM192 (chr4), ATXN1 (chr6), APBA2 (chr15), CTNND2 (chr5), upstream of HELB (chr12), at a position that is just upstream of CHRNA5 and downstream of PSMA4 (chr15), as well as at certain intergenic regions in certain chromosomes. Intergenic insertions were detected which included regions 13 Kb downstream of SMPX and 34 Kb upstream of KLHL34 in X chromosome, 10 Kb upstream of ELFN1 and 82 Kb downstream of TFAMPI (chr7). Except for TFAMP1, all other genes were found to be associated with epithelial cancer (p-value=2.11E-03) (FIG. 7E).

Similar to the HHV3 data, a specific region of the HHV7 genome was integrated at multiple sites in the chromosomes (FIGS. 7B-7C). The CDS of the U30 gene of HHV7, encoding the tegument protein UL37 that helps in virion morphogenesis was found to be integrated at the intronic or intergenic region of certain chromosomes. HHV3 insertions were detected at the intronic regions of ZNF225 (chr19), TENM1 (chrX) and HTR2C (chrX), and also at certain intergenic regions, some of which are less than 35 Kb from the affected genes. Therefore, this may have an effect on promoting or suppressing the transcription of those genes. For example, insertions were detected 17 Kb downstream of RASSF6 and 26 Kb downstream of LOC728040 in chromosome 4; 32 Kb downstream of GDAP1 (chr8); 11 Kb downstream of USP15 and 46 Kb upstream of MON2 (chrl 2); 35 Kb downstream of GABRA2 and 90 Kb upstream of GABRG1 (chr4). Except for LOC728040, the other genes having HHV7 genomic insertions at or in their proximity were seen to be significantly associated with adenocarcinoma (p value=2.33E-04) (FIG. 7E).

### Example 10: Insertions Detected for Retrovirus, Hepadnavirus, Yaba Monkey Tumor Virus and Frog Virus3

Among the other viral insertions detected were HTLV-2, whose genomic region encoding gag-pro-pol was detected at the intronic region of CCDC88C (chr14). The 3′UTR region of HCV was detected at the intronic, intergenic as well as downstream of certain genes in a number of chromosomes. Insertion was detected at the intronic region of RBM4 (chr11), known to be associated with cancer and ncRNA SMG1P5 (chr16), downstream of TINAGL1 (chrl) and LOC339807 (chr2) and at an intergenic region that is 30 Kb upstream of ZNF846 and 11 Kb downstream of FBXL12 in chromosome 19. Interestingly, Yaba Monkey Tumor Virus (YMTV) genomic sequences encoding the G protein-coupled chemokine receptor-like protein were detected at the intergenic region of a number of genes in chromosome 5. Also detected were Alloherpesviridae genomic sequence (Frog virus 3) insertions in host chromosomes. CDS of FV3gorf8R gene encoding the largest sub-unit of DNA-dependent RNA polymerase II of Frog virus 3 was inserted at the intronic region of FAT3 gene (chrl 1), upstream of PTGDR gene (chr14), 86 Kb downstream of C15orf59-AS1 and 18 Kb upstream of TBC1D21 gene (chr15). FAT3 gene and PTGDR gene, both are seen to be associated significantly (p-value=8.41E-04) with esophageal adenocarcinoma by IPA analysis.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of detecting ovarian cancer in a tumor tissue sample from a subject, the method comprising:
hybridizing a detectably-labeled nucleic acid from the tumor tissue sample to a PathoChip array to generate a first hybridization pattern;
hybridizing a detectably-labeled nucleic acid from a reference sample to a PathoChip array to generate a second hybridization pattern, wherein the reference sample is from an otherwise identical non-tumor tissue from a subject;
comparing the first and second hybridization patterns, wherein when the first hybridization pattern is substantially a microbial hybridization signature and the second hybridization pattern is substantially not a microbial hybridization signature, ovarian cancer is detected in the tumor tissue sample.

2. The method of claim 1, wherein the microbial hybridization signature is generated by hybridization of the detectably-labeled nucleic acid from the tumor tissue sample to at least three nucleic acid probes on the PathoChip, wherein the probes are from microbes selected from the group consisting of: Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

3. The method of claim 2, wherein the at least three nucleic acid probes are selected from the group consisting of SEQ ID NOs: 1-94.

4. A method of detecting ovarian cancer in a tumor tissue sample from a subject, the method comprising:
hybridizing a detectably-labeled nucleic acid from the tumor tissue sample to a first microarray comprising at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria to generate a first hybridization pattern;
hybridizing a detectably-labeled nucleic acid from a reference sample to a second microarray comprising at least three nucleic acid probes from microbes selected from the group consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria to generate a second hybridization pattern, wherein the reference sample is from an otherwise identical non-tumor tissue from a subject;
comparing the first and second hybridization patterns, wherein when the first hybridization pattern is substantially a microbial hybridization signature and the second hybridization pattern is substantially not a microbial hybridization signature, ovarian cancer is detected in the tumor tissue sample.

5. The method of claim 4, wherein the at least three nucleic acid probes are selected from the group consisting of SEQ ID NOS: 1-94.

6. The method of claim 1, wherein the tumor tissue sample is selected from the group consisting of a biopsy, formalin-fixed, paraffin-embedded (FFPE) sample, or non-solid tumor.

7. The method of claim 1, wherein the subject is human.

8. The method of claim 1, wherein the detectably-labeled nucleic acid is labeled with a fluorophore, radioactive phosphate, biotin, or enzyme.

9. The method of claim 8, wherein the fluorophore is Cy3 or Cy5.

10. The method of claim 1, further comprising wherein when oral ovarian cancer is detected in the tumor tissue sample from a subject, the subject is provided with a treatment for ovarian cancer.

11. The method of claim 10, wherein the treatment comprises surgery, chemotherapy, or radiotherapy.

12. A composition comprising at least three nucleic acid probes selected from the group consisting of SEQ ID NOS: 1-94.

13. A microarray comprising at least three nucleic acid probes selected from the group consisting of SEQ ID NOS: 1-94.

14. The microarray of claim 13, wherein the nucleic acid probes are selected from about 10 to about 30 microbes and comprise about 3 to about 5 probes per microbe.

15. A microarray comprising at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

16. The microarray of claim 13, wherein the microarray is a biochip, glass slide, bead, or paper.

17. A kit comprising at least three nucleic acid probes selected from the group consisting of SEQ ID NOS: 1-94, and instructional material for use thereof.

18. A kit comprising a microarray comprising at least three nucleic acid probes selected from the group consisting of SEQ ID NOS: 1-94, and instructional material for use thereof.

19. A kit comprising a microarray comprising at least three nucleic acid probes selected from the group of microbes consisting of Anelloviridae, Astroviridae, Birnaviridae, Bornaviridae, Caliciviridae, Hepadnaviridae, Iridoviridae, Paramyxoviridae, Rhabdoviridae, Togaviridae Abiotrophia, Aeromonas, Agrobacterium, Anaplasma, Arcobacter, Bacillus, Bacteroides, Bartonella, Brucella, Burkholderia, Campylobacter, Chlamydia, Chlamydophila, Corynebacterium, Coxiella, Enterococcus, Erysipelothrix, Flavobacterium, Francisella, Fusobacterium, Geobacillus, Helicobacter, Klebsiella, Lactobacillus, Lactococcus, Legionella, Leptospira, Listeria, Methylobacterium, Mycoplasma, Neisseria, Orientia, Pasteurella, Pediococcus, Peptoniphilus, Porphyromonas, Prevotella, Propionibacterium, Proteus, Pseudomonas, Rickettsia, Shewanella, Shigella, Sphingomonas, Staphylococcus, Stenotrophomonas, Streptobacillus, Treponema, Ureaplasma, Vibrio, Wolbachia, Yersinia, Acremonium, Ajellomyces, Aspergillus, Candida, Cladosporium, Coccidioides, Cryptococcus, Cunninghamella, Issatchenkia, Nosema, Paracoccidioides, Penicillium, Pleistophora, Pneumocystis, Rhizomucor, Rhizopus, Rhodotorula, Trichophyton, Ancylostoma, Anisakis, Armiilifer, Ascaris, Babesia, Balantidium, Bipolaris, Blastocystis, Capillaria, Dicrocoelium, Dipylidium, Echinococcus, Echinostoma, Entamoeba, Enterobius, Hartmannella, Heteroconium, Hymenolepis, Leishmania, Loa, Metagonimus, Necator, Onchocerca, Plasmodium, Sarcocystis, Schistosoma, Strongyloides, Toxascaris, Toxocara, Trichomonas, Trichuris and Wuchereria.

20. The kit of claim 17, wherein the nucleic acid probes are selected from between about 10 to about 30 microbes and comprise about 3 to about 5 probes per microbe.
